(19) United States
(12) Patent Application Publication
(10) Pub. No.: US 2024/0352398 A1
(43) Pub. Date: Oct. 24, 2024

(54) METHODS AND SYSTEMS FOR INCREASING HOMOGENEITY OF AN EXTRA-PARTICLE AERIAL MYCELIUM

(71) Applicant: Ecovative Design LLC, Green Island, NY (US)

(72) Inventors: Benjamin Michael Sword, Somerville, MA (US); Alex Carlton, Troy, NY (US); Asa Trench Synder, Petersburg, NY (US); Jacob Michael Winiski, Troy, NY (US)

(21) Appl. No.: 18/642,596

(22) Filed: Apr. 22, 2024

Related U.S. Application Data
(60) Provisional application No. 63/498,003, filed on Apr. 24, 2023.

(51) Int. Cl. C12M 1/36; C12M 1/00; C12M 1/34; C12M 1/42; C12N 1/14; C12R 1/645

(52) U.S. Cl. CPC C12M 41/48; C12M 29/06; C12M 35/02; C12M 41/12; C12M 41/34; C12M 41/40; C12N 1/14; C12R 2001/645

(57) ABSTRACT

A method of growing an extra-particle aerial mycelium employs electrostatically charged mist to control mist deposition uniformity in a growth environment, and on a growth matrix (or growing extra-particle aerial mycelium on a growth matrix) comprising nutritive substrate and a fungus in a growth environment with a predetermined environment of humidity, temperature, carbon dioxide, and oxygen. Control of mist deposition by electrostatically charging mist allows for control of the morphology and uniformity of the extra-particle aerial mycelium produced, independent of airflow uniformity, growth environment size, and structural obstacles that may be present within a growth environment. Control of mist deposition by electrostatically charging mist also offers the potential for targeted mist application where it might be needed most for fungal organism growth.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field

The present application relates to systems, apparatus, and methods for growing aerial mycelium material, and in particular, for controlling mist deposition, dispersion, and uniformity on a growth matrix comprising nutritive substrate and a fungus contained in a growth environment. The present application also relates to improved systems, apparatus, and methods for creating homogeneous aerial mycelial growth.

### Description

Application of mist to the growth surface of mycelial growth matrices and actively growing mycelium has been demonstrated to be a significant component of aerial mycelium growth production, especially in solid state fermentation contexts. Solid-state fermentation processes may generally be described as fungal fermentation processes that utilize solid particulate matter or semi-solid particulate, slurry-like matter as the basis of nutritive inputs for fungal mycelial growth. Solid particulate or semi-solid, slurry-like matter may be held or supported in open beds, racks, tray-like containers, or even on webs or netting (which may be moveable) during the growth of mycelium in a growth environment.

For example, the uniformity of mist deposition can influence the morphology and consistency of aerial mycelium tissue that is grown in a growth environment. While, to date, mist deposition has been monitored through various visual means and has been manipulated by controlling airflow rates and directions within a growth environment, such controls lack precise control of growth uniformity. For instance, extra-particle aerial mycelium often grows to form bulbous structures. Non-uniform mist deposition may result in heterogeneous bulb formation or the lack of mycelial growth at particular locations within a growth environment. The lack of uniform growth may be the result of hindered airflow in and around racks or other support structures contained in growth environments, resulting in heterogeneous mist deposition. Lack of uniform growth may also be accentuated by the size of growth environments, where larger-sized growth environments create additional challenges for providing airflow and mist uniformity.

Current state-of-the-art mist deposition varies with airflow eccentricity, and uniformity of mist deposition is dependent on airflow eccentricity and gravity. Consequently, irregularities that stem from airflow management apparatus (e.g., duct placement and HVAC fan units) and/or physical structural impediments within a growth environment (e.g., racks or other support structures for holding growth matrices or growing mycelium in a growth environment around which airflow needs to travel) have a direct effect on the uniformity of extra-particle aerial mycelium.

Thus, there is a need for improved methods of controlling mist deposition uniformity in aerial mycelium growth systems, and for apparatus which assist in facilitating uniform mist deposition in aerial mycelium growth systems, regardless of the size of the growth environment and independent of airflow within the growth environment. There is a further need for controlling mist deposition uniformity that overcomes challenges associated with physical hindrances within a growth environment. There is still a further need for decoupling mist deposition control from airflow control. There is still a further need for methods and systems for targeted mist deposition.

### SUMMARY OF THE INVENTION

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method of growing extra-particle aerial mycelium (and the eventual aerial mycelium) can include: maintaining a growth environment configured to produce extra-particle aerial mycelium; and introducing a plurality of electrostatically charged mist particles into the growth environment.

In some aspects, the method can further include: providing a growth matrix including a nutritive substrate and a fungus; and placing the growth matrix into the growth environment.

In some aspects, the method can further include incubating the growth matrix in the growth environment for an incubation time period sufficient for the fungus to digest the nutritive substrate and produce the extra-particle aerial mycelium.

In some aspects, the method can include the plurality of electrostatically charged mist particles including at least one of: water, fertilizers, nutrients, growth factors, hormones, disinfectants, cleaning solutions, antioxidants, pesticides, integrated pest management sprays, fine powders, microorganisms, dyes, flavonoids, and terpenes.

In some aspects, the method can include introducing the plurality of electrostatically charged mist particles into the growth environment including introducing the plurality of electrostatically charged mist particles periodically during an incubation time period.

In some aspects, the method can include introducing the plurality of electrostatically charged mist particles into the growth environment including introducing the plurality of electrostatically charged mist particles during a portion of the incubation time period.

In some aspects, the method can include introducing the plurality of electrostatically charged mist particles into the growth environment including: introducing a first portion of the plurality of electrostatically charged mist particles during a first time portion of an incubation time period, including the first portion of the plurality of electrostatically charged mist particles having a first charge during the first time portion; and introducing a second portion of the plurality of electrostatically charged mist particles during a second time portion of the incubation time period, including the second portion of the plurality of electrostatically charged mist particles having a second charge during the second time portion.

In some aspects, the method can include the second charge including one of: a neutral charge or an opposite charge of the first charge.

In some aspects, the method can include the plurality of electrostatically charged mist particles being between 0.01 μm to 100 μm in diameter, alternatively between 1.0 μm to 50 μm in diameter.

In some aspects, the method can include introducing the plurality of electrostatically charged mist particles into the growth environment further including passing a stream of mist particles through a charge generator.

In some aspects, the method can include the charge generator including an electromagnetic field.

In some aspects, the method can further include generating a voltage with the charging generator between 0.01 kV to 50.0 kV.

In some aspects, the method can include the growth environment being configured to maintain one or more of: a temperature, a humidity, an airflow rate, an oxygen concentration, a carbon dioxide concentration, and/or a pressure.

In some aspects, the method can further include generating an airflow in the growth environment.

In some aspects, the method can include the electrostatically charged mist particles being homogeneously deposited across the growth matrix.

In some aspects, the method can include, as a result of the homogeneous deposition of the electrostatically charged mist particles, the growing extra-particle aerial mycelium growing homogeneously.

In some aspects, the method can include growing the extra-particle aerial mycelium via solid state fermentation, including the substrate, the nutritive substrate, the growth medium, and/or the growth matrix including one or more solids.

In some aspects, the method can further include introducing a voltage to at least a portion of the growth matrix, thereby imparting a charge to the at least portion of the growth matrix.

In some other aspects, the method can further include introducing a voltage to the support structure holding the growth matrix, thereby imparting a charge to a location in the growth environment where electrostatically charged mist is desired to be deposited.

In some other aspects, the method can further include introducing a voltage to an adjacent material or structure within the growth environment (e.g., adjacent to the growth matrix), thereby imparting a charge to a location in the growth environment adjacent to where electrostatically charged mist is desired to be deposited.

In one aspect, the method can further include the location in the growth environment adjacent to where electrostatically charged mist is desired to be deposited, the location being within 5 ft of the growth matrix, 3 ft of the growth matrix, 1 ft of the growth matrix, or 6 inches of the growth matrix.

In some aspects, the method can include pulsing a charge generator to create a linear motion to move airborne masses of mist in a linear direction, including a linear motion along a series of electrical plates or electrical surfaces placed in serial and in electrical communication with the charge generator such that the charge generator can pulse on and off each of the series of electrical plates or surfaces individually, or in groups, along the series, thereby propelling the mass of airborne mist across the length of the series of electrical plates or surfaces.

In another aspect, a system for producing an extra-particle aerial mycelium, the system can include: a growth environment; an electrostatically charged misting system, including, an outlet configured to introduce a plurality of mist particles into the growth environment, a charge generator disposed at a distal end of the outlet, the charge generator configured to impart an electrostatic charge to the plurality of mist particles to generate a plurality of electrostatically charged mist particles; and a processor configured to introduce the plurality of mist particles into the growth environment.

In some aspects, the system can include the growth environment further comprising a growth matrix received within the growth environment, the growth matrix including a nutritive substrate and a fungus.

In some aspects, the system further can include the plurality of electrostatically charged mist particles, including at least one of: water, fertilizers, nutrients, growth factors, hormones, disinfectants, cleaning solutions, antioxidants, pesticides, integrated pest management sprays, fine powders, microorganisms, dyes, flavonoids, and terpenes.

In some aspects, the system can further include the processor being further configured to periodically introduce the plurality of electrostatically charged mist particles.

In some aspects, a system can further include the processor being further configured to: introduce a first portion of the plurality of electrostatically charged mist particles during a first time portion, including the first portion of the plurality of electrostatically charged mist particles having a first charge during the first time portion; and introduce a second portion of the plurality of electrostatically charged mist particles during a second time portion, including the second portion of the plurality of electrostatically charged mist particles having a second charge during the second time portion.

In some aspects, the system can further include the plurality of electrostatically charged mist particles being between 0.01 μm to 100 μm in diameter, alternatively between 1.0 μm to 50 μm in diameter.

In some aspects, the system can include the outlet being further configured to pass a stream of uncharged mist particles through the charge generator.

In some aspects, the system including the charge generator including an electromagnetic field.

In some aspects, the system can include the charge generator being configured to generate a voltage between 0.01 kV to 50.0 kV.

In some aspects, the system can include the charge generator including one of: an induction electrostatic charging ring, a conduction charging unit, a corona charging unit, or a high velocity air sprayer.

In some aspects, the system can include the growth environment being configured to maintain one or more of: a temperature, a humidity, an airflow rate, an oxygen concentration, a carbon dioxide concentration, and/or a pressure.

In some aspects, the system can include the growth environment being further configured to generate airflow within the growth environment.

In some aspects, the system can include the extra-particle aerial mycelium being adapted to grow in a solid state fermentation system, including the substrate, the nutritive substrate, the growth medium, and/or the growth matrix comprising one or more solids.

In some aspects, the system can further include introducing a voltage to at least a portion of the growth matrix thereby imparting a charge in the growth matrix, wherein the charge may attract electrostatically charged mist.

In some other aspects, the system can further include introducing a voltage to the support structure holding the growth matrix, thereby imparting a charge to a location in the growth environment where electrostatically charged mist is desired to be deposited.

In some other aspects, the system can further include introducing a voltage to an adjacent material or structure within the growth environment (e.g. adjacent to the growth matrix), thereby imparting a charge to a location in the growth environment adjacent to where electrostatically charged mist is desired to be deposited.

In one aspect, the system can further include the location in the growth environment adjacent to where electrostatically charged mist is desired to be deposited, the location being within 5 ft of the growth matrix, 3 ft of the growth matrix, 1 ft of the growth matrix, or 6 inches of the growth matrix.

In some aspects, the system can include a plurality of charge generators, the charge generators positioned linearly and configured to create a linear motion to move airborne masses of mist in a linear direction, including a linear motion along a series of electrical plates or electrical surfaces placed in serial and in electrical communication with the charge generator such that the charge generator can pulse on and off each of the series of electrical plates or surfaces individually, or in groups, along the series, thereby propelling the mass of airborne mist across the length of the series of electrical plates or surfaces.

In another aspect of the methods and systems described, either the application of electrostatically charged mist or electrostatically charged mist with accompanying application of voltage to either the growth matrix, support structure, or adjacent material or structure location, is also accompanied by a coordination in airflow within the growth environment.

In yet another alternative embodiment, at least a portion of the charges of the electrostatically charged mist can include the same charge as that of at least a portion of either the charged growth matrix, charged support structure, or charged adjacent material or structure (so as to prevent application of mist to certain locations, by charge repulsion).

In still another alternative embodiment, at least a portion of the charges of the electrostatically charged mist of the opposite charge as that of at least a portion of either the charged growth matrix, can include the charged support structure, or charged adjacent material or structure.

In another aspect, a method of growing extra-particle aerial mycelium can include: maintaining a growth environment including a growth matrix, the growth matrix including a substrate and configured to produce extra-particle aerial mycelium; and introducing a plurality of electrostatically charged mist particles into the growth environment, including the plurality of electrostatically charged mist particles being electrostatically charged such that they repel one another, including the electrostatic charged mist particles being sufficiently charged to allow the plurality of electrostatic charged mist particles to uniformly deposit on the growth matrix surface, or a portion thereof, despite airflow conditions and physical hindrances within the growth environment, and including the growth matrix further including a target deposition surface selected from the substrate surface or an extra-particle aerial mycelium surface growing on the substrate.

In some aspects, the method can include uniform deposition of the plurality of electrostatically charged mist particles being unaffected by fluctuations in the airflow conditions in the growth environment.

In some aspects, the method can include monitoring the airflow conditions such that fluctuations in the airflow conditions do not affect uniform mist deposition on the target deposition surface, or portion thereof.

In some aspects, the method can include growing extra-particle aerial mycelium including: maintaining a growth environment configured to produce extra-particle aerial mycelium; and introducing a plurality of electrostatically charged mist particles into the growth environment.

In some aspects, the method further can include: providing a growth matrix including a nutritive substrate and a fungus; and placing the growth matrix into the growth environment.

In some aspects, the method further can include incubating the growth matrix in the growth environment for an incubation time period sufficient for the fungus to digest the nutritive substrate and to produce the extra-particle aerial mycelium.

In some aspects, the method can include providing the growth matrix further including: providing at least one container defining a cavity containing the growth matrix, and including placing the at least one container into a growth environment.

In some aspects, the method can include the plurality of electrostatically charged mist particles including reverse osmosis water.

In some aspects, the method can include the plurality of electrostatically charged mist particles including at least one of: water, fertilizers, nutrients, growth factors, hormones, disinfectants, cleaning solutions, antioxidants, pesticides, integrated pest management sprays, fine powders, microorganisms, dyes, flavonoids, and terpenes.

In some aspects, the method can include introducing the plurality of electrostatically charged mist particles into the growth environment including introducing the plurality of electrostatically charged mist particles periodically during an incubation time period.

In some aspects, the method can include introducing the plurality of electrostatically charged mist particles into the growth environment including introducing the plurality of electrostatically charged mist particles during a portion of the incubation time period.

In some aspects, the method can include introducing the plurality of electrostatically charged mist particles into the growth environment including: introducing the plurality of electrostatically charged mist particles during a first time portion of an incubation time period, including the plurality of electrostatically charged mist particles having a first charge during the first time portion; and introducing the plurality of electrostatically charged mist particles during a second time portion of the incubation time period, including the plurality of electrostatically charged mist particles having a second charge during the second time portion.

In some aspects, the method can include the second charge being one of: a neutral charge or an opposite charge of the first charge, in an alternative, the same charge of the first charge.

In some aspects, the method can include the plurality of electrostatically charged mist particles being between 0.01 μm to 100 μm in diameter, alternatively between 1.0 μm to 50 μm in diameter.

In some aspects, the method can include the plurality of electrostatically charged mist particles comprising a charge to mass ratio within a range of 0.05 to 2.0 C/kg.

In some aspects, the method can include the plurality of electrostatically charged mist particles comprising a negative charge.

In some aspects, the method can include the plurality of electrostatically charged mist particles comprising a positive charge.

In some aspects, the method can include introducing the plurality of electrostatically charged mist particles into the growth environment further including passing a stream of mist particles through a charge generator.

In some aspects, the method can include the stream of mist particles being delivered through one or more of: an ultrasonic misting spray nozzle, an ultrasonic misting puck, a high-pressure misting nozzle, or a high velocity air sprayer.

In some aspects, the method can include the charge generator including an electromagnetic field.

In some aspects, the method further can include generating a voltage at the charge generator between 0.01 kV to 50.0 kV.

In some aspects, the method can include the charge generator including an induction electrostatic charging ring, one or more conduction charging units, one or more corona charging units, or one or more high velocity air sprayers.

In some aspects, the method can include the growth environment being configured to maintain one or more of: a temperature, a humidity, an airflow rate, an oxygen concentration, a carbon dioxide concentration, and/or a pressure.

In some aspects, the method further can include generating an airflow in the growth environment.

In some aspects, the method further can include coordinating airflow velocity, direction, or a combination thereof, with application of the electrostatically charged mist within the growth environment.

In some aspects, the method can include the airflow being generated by one or more of: an HVAC unit, one or more ceiling fans, one or more wall-mounted fans, one or more oscillating fans, one or more vertical circulation fans, one or more blower fans, one or more squirrel cage fans, or one or more barrel fans.

In some aspects, the method further can include monitoring the plurality of electrostatically charged mist particles to determine their locations or quantity within the growth environment.

In some aspects, the method further can include adjusting a production rate or an electrostatic mist target location of electrostatically charged mist particles within the growth environment in response to the monitoring.

In some aspects, the method further can include adjusting aspects of the growth environment in response to said monitoring and the determined location of the electrostatically charged mist particles.

In some aspects, the method further can include fine tuning one or more environmental conditions in the growth environment in response to the monitoring, the one or more environmental conditions selected from humidity, temperature, gas content, airflow, and mist deposition.

In some aspects, the method further can include: monitoring extra-particle aerial mycelial growth or aspects of the growth environment; and adjusting either a production rate or a target location of the plurality of electrostatically charged mist particles based on said monitoring.

In some aspects, the method can further can include introducing a voltage to at least a portion of the growth matrix, alternatively, the support structure holding the growth matrix, alternatively an adjacent material or structure adjacent to the growth matrix, thereby imparting a charge to the at least portion of the growth matrix, or alternatively, the support structure holding the growth matrix, or alternatively an adjacent material or structure adjacent to the growth matrix.

In one aspect, the method can further include the location in the growth environment adjacent to where electrostatically charged mist is desired to be deposited, the location being within 5 ft of the growth matrix, 3 ft of the growth matrix, 1 ft of the growth matrix, or 6 inches of the growth matrix.

In some aspects, the method can include pulsing a charge generator to create a linear motion to move airborne masses of mist in a linear direction, including a linear motion along a series of electrical plates or electrical surfaces placed in serial and in electrical communication with the charge generator such that the charge generator can pulse on and off each of the series of electrical plates or surfaces individually, or in groups, along the series, thereby propelling the mass of airborne mist across the length of the series of electrical plates or surfaces.

In another aspect, a system for producing an extra-particle aerial mycelium, the system can include: a growth environment; an electrostatically charged misting system, including, an outlet configured to introduce a plurality of mist particles into the growth environment, a charge generator disposed at a distal end of the outlet, the charge generator configured to impart an electrostatic charge to the plurality of mist particles to generate a plurality of electrostatically charged mist particles; and a processor configured to introduce the plurality of mist particles into the growth environment.

In some aspects, the system can include the growth environment being configured to receive a growth matrix, the growth matrix including a nutritive substrate and a fungus.

In some aspects, the system can include the growth environment being a growth environment configured to incubate the growth matrix for an incubation time period sufficient for the fungus to digest the nutritive substrate and produce the extra-particle aerial mycelium.

In some aspects, the system can include the growth matrix being disposed in at least one container defining a cavity containing the growth matrix.

In some aspects, the system can include the growth matrix being disposed in (or held upon) a support structure, the support structure selected from a tray, a bed, an open rail shelf or rack, a table, a relatively horizontal surface of either a solid or perforated material, and having no side walls.

In some aspects, the system can include the plurality of electrostatically charged particles including reverse osmosis water or, alternatively, tap water.

In some aspects, the system can include the plurality of electrostatically charged mist particles including at least one of: water, fertilizers, nutrients, growth factors, hormones, disinfectants, cleaning solutions, antioxidants, pesticides, integrated pest management sprays, fine powders, microorganisms, dyes, flavonoids, and terpenes.

In some aspects, the system can include the processor being further configured to periodically introduce the plurality of electrostatically charged mist particles.

In some aspects, the system can include the processor being further configured to: introduce the plurality of electrostatically charged mist particles during a first time portion, including the plurality of electrostatically charged mist particles having a first charge during the first time portion; and introduce the plurality of electrostatically charged mist particles during a second time portion, including the plurality of electrostatically charged mist particles having a second charge during the second time portion.

In one aspect, the system can include the two charges, the two charges being a same charge.

In another aspect, the system can include the two charges, the two charges being different, including one charge being opposite or neutral from the other charge.

In some aspects, the system can include the second charge being one of: no charge or an opposite charge of the first charge.

In some aspects, the system can include the plurality of electrostatically charged mist particles being between 0.01 μm to 100 μm in diameter, alternatively between 1.0 μm to 50 μm in diameter.

In some aspects, the system can include the plurality of electrostatically charged mist particles comprising a charge to mass ratio within a range of 0.05 to 2.0 C/kg.

In some aspects, the system can include the plurality of electrostatically charged mist particles comprising a negative charge.

In some aspects, the system can include the plurality of electrostatically charged mist particles comprising a positive charge.

In some aspects, the system including the outlet being further configured to pass a stream of uncharged mist particles through the charge generator.

In some aspects, the system can include the outlet being one of: an ultrasonic misting spray nozzle, an ultrasonic misting puck, a high-pressure misting nozzle, or a high velocity air sprayer.

In some aspects, the system can include the charge generator including an electromagnetic field.

In some aspects, the system can include the charge generator being configured to generate a voltage between 0.01 kV to 50.0 kV.

In some aspects, the system can include the charge generator being one of: an induction electrostatic charging ring, a conduction charging unit, a corona charging unit, or a high velocity air sprayer.

In some aspects, the system can include the growth environment being configured to maintain one or more of: a temperature, a humidity, an airflow rate, an oxygen concentration, a carbon dioxide concentration, or a pressure.

In some aspects, the system can include the growth environment being further configured to generate airflow within the growth environment.

In some aspects, the system can include the growth environment including an airflow generator configured to generate the airflow within the growth environment, including the airflow generator being one or more of: an HVAC unit, one or more ceiling fans, one or more wall-mounted fans, one or more oscillating fans, one or more vertical circulation fans, one or more blower fans, one or more squirrel cage fans, or one or more barrel fans.

In some aspects, the system can include the growth environment including an airflow generator and the electrostatic mist generator, including the airflow generator and the electrostatic mist generator being synchronized so as to produce a homogeneous distribution and/or deposition of electrostatically charged mist upon the growth matrix (or growing extra-particle aerial mycelium).

In some aspects, the system can include a plurality of charge generators, the charge generators positioned linearly and configured to create a linear motion to move airborne masses of mist in a linear direction, including a linear motion along a series of electrical plates or electrical surfaces placed in serial and in electrical communication with the charge generator such that the charge generator can pulse on and off each of the series of electrical plates or surfaces individually, or in groups, along the series, thereby propelling the mass of airborne mist across the length of the series of electrical plates or surfaces.

In some aspects, the method further can include monitoring electrostatically charged mist particles to determine either their locations or quantity within the growth environment.

In some aspects, the method further can include adjusting levels and/or locations of electrostatically charged mist particles within said growth environment in response to the monitoring.

In some aspects, the method further can include adjusting aspects including monitoring extra-particle aerial mycelium or aspects of the growth environment, and adjusting either the level of or location of electrostatically charged mist particles in response to the monitoring.

In some aspects, the method can include the growth environment including solid-state substrate.

In another aspect, a method of growing extra-particle aerial mycelium, can include: maintaining a growth environment with a growth matrix configured to produce extra-particle aerial mycelium; and introducing a plurality of electrostatically charged mist particles into the growth environment, including the electrostatically charged mist particles being charged such that they repel one another in the growth environment.

In some aspects, a method of growing extra-particle aerial mycelium further can include providing the growth matrix in or on a support structure within the growth environment and charging either the growth matrix or support structure or the combination thereof, such that electrostatically charged mist particles are attracted to either the growth matrix, the support structure or both of the growth matrix and the support structure.

In some aspects, a method of growing extra-particle aerial mycelium further can include providing the growth matrix in or on a support structure within the growth environment and charging the growth matrix or a portion thereof, the support structure or a portion thereof, or an adjacent material or structure in the growth environment (or a portion thereof) or some combination thereof, such that electrostatically charged mist particles are attracted to either the growth matrix, the support structure, an adjacent material or structure adjacent to the growth matrix, or some combination thereof.

In some aspects, the method can include the electrostatically charged mist particles being homogeneously deposited across the growth matrix.

In some aspects, the method can include, as a result of the homogeneous deposition of the electrostatically charged mist particles, the growing extra-particle aerial mycelium growing homogeneously (e.g. little to no occurrence of bulbous surface structures, regular occurrence of surface structures on the extra particle aerial mycelium).

In some aspects, the method can include the electrostatically charged mist particles being deposited on only a portion of the growth matrix.

In some aspects, the method can include the electrostatically charged mist particles being attracted to only a portion of the growth matrix.

In some aspects, the method can include selectively attracting the electrostatically charged mist particles to only portions of the growth matrix.

In some aspects, the method further can include electrostatically attracting the electrostatically charged mist particles to the growth matrix.

In another aspect, a method of growing extra-particle aerial mycelium can include: maintaining a growth environment including a growth matrix, the growth matrix including a substrate and configured to produce extra-particle aerial mycelium; and introducing a plurality of electrostatically charged mist particles into the growth environment, including the plurality of electrostatically charged mist particles being electrostatically charged such that they repel one another, including the electrostatically charged mist particles being sufficiently charged to allow the plurality of electrostatically charged mist particles to uniformly deposit on the growth matrix surface, or a portion thereof, despite airflow conditions and physical hindrances within the growth environment, and including the growth matrix further including a target deposition surface selected from the substrate surface or an extra-particle aerial mycelium surface growing on the substrate.

In some aspects, the method can include uniform deposition of the plurality of electrostatically charged mist particles being unaffected by fluctuations in the airflow conditions in the growth environment.

In some aspects, the method can include monitoring the airflow conditions such that fluctuations in the airflow conditions do not affect uniform mist deposition on the target deposition surface, or portion thereof.

In some aspects, the method can further include introducing a voltage to at least a portion of the growth matrix, thereby imparting a charge to the at least portion of the growth matrix.

In some aspects, the method can further include introducing a voltage to at least a portion of the support structure holding the growth matrix.

In some aspects, the method can further include introducing a voltage to at least a portion of an adjacent material or structure that is adjacent to the growth matrix within the growth environment.

In some aspects, the method can include pulsing a charge generator to create a linear motion to move airborne masses of mist in a linear direction, including a linear motion along a series of electrical plates or electrical surfaces placed in serial and in electrical communication with the charge generator such that the charge generator can pulse on and off each of the series of electrical plates or surfaces individually, or in groups, along the series, thereby propelling the mass of airborne mist across the length of the series of electrical plates or surfaces.

Therefore, the present application relates to methods and systems for increasing homogeneity of extra-particle aerial mycelium growth via electrostatically charged mist facilitating uniform mist deposition, and apparatus and methods for facilitating unform mist deposition and monitoring of such.

All of these embodiments are intended to be within the scope of the invention herein disclosed. These and other embodiments of the present invention will become readily apparent to those skilled in the art from the following detailed description of the embodiments having reference to the attached figures, the invention not being limited to any particular embodiment(s) disclosed.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix suitable to support extra-particle aerial mycelial growth.

FIG. 1B illustrates an embodiment of extra-particle aerial mycelial growth extending from the growth matrix of FIG. 1A.

FIG. 1C illustrates an embodiment in which the extra-particle aerial mycelial growth and growth matrix in FIG. 1B have been divided to form a separated aerial mycelium and depleted growth matrix.

FIG. 1D illustrates an embodiment of the separated aerial mycelium in FIG. 1C that has been cut to form an aerial mycelium panel.

FIG. 2 illustrates an isometric view of a system for cultivating an extra-particle aerial mycelium.

FIG. 3 illustrates an exemplary embodiment of a charged misting system.

FIG. 4 illustrates an exemplary system for producing an extra-particle aerial mycelium.

FIG. 5 illustrates an exemplary process for producing extra-particle aerial mycelium.

FIG. 6A illustrates an experimental setup demonstrating an exemplary embodiment of a charged misting system.

FIG. 6B illustrates a target, as shown in FIG. 6A, at a greater level of detail.

FIG. 7A is a box-whisker plot demonstrating mist deposition rates split between the control targets and charged targets within their various electrical treatments.

FIG. 7B is a chart demonstrating a t-test statistical analysis between the average mist deposition measurements of the grounded and charged targets in the electrical treatments shown in FIG. 7A.

FIG. 8A is a chart demonstrating the difference in mist deposition rates between a ground electrical treatment and a high voltage electrical treatment.

FIG. 8B is a chart demonstrating t-test analysis of the difference in mist deposition rates by electrical treatment shown in FIG. 7A.

FIG. 9A is a chart demonstrating average effect size of average mist deposition rates within a high voltage treatment of the mist plotted by the starting relative humidity (RH) value.

FIG. 9B is a chart illustrating a linear fit statistical analysis of the chart shown in FIG. 9A.

### DETAILED DESCRIPTION

The uniform deposition of a mist within a growth environment can be important for growth and production of aerial mycelium, for example, with desired morphological characteristics. Such morphological characteristics may include the elimination of bulbous formations in an aerial mycelium panel. Currently, mist deposition is primarily reliant on the uniformity of airflow, which acts as a vector for mist transference and deposition.

The following discussion presents detailed descriptions of the several embodiments of apparatus, systems, and methods for growing mycelium, such as extra-particle aerial mycelium, for example, through introduction of electrostatically charged mist within a growth environment, and using such apparatus, systems, and methods for monitoring and facilitating uniformity of mist dispersion and mist deposition, and thereafter adjusting mist inputs/trajectories accordingly, to account for variances observed in mist deposition uniformity (e.g., the mist itself, or alternatively the growing extra-particle aerial mycelium product). The apparatus, systems, and methods provide electrostatically charged mist liquid and electrostatically charged additive-containing liquid to ultimately adjust/control and monitor mist deposition uniformity on a growth matrix (or growth media or substrate) comprising nutritive substrate and a fungus. The mist can be deposited on the growth matrix (or growth media or substrate) in a growth environment (e.g., a growth environment, growth chamber, growth room, or bench top bioreactor) with a predetermined environment of humidity, temperature, and gas content (e.g., carbon dioxide, and oxygen, at least). These embodiments are not intended to be limiting, and modifications, variations, combinations, etc., are possible and within the scope of this disclosure.

Mist deposition rates may change over time when growing extra-particle aerial mycelium in a large growth environment due to organism growth cycles, the temporal differences in environmental conditions and air conditioning strategies, including, but not limited to, airflow control. For example, while programmed mist deposition rates may be known for any given area of a growth environment, realized mist deposition rates at specific locations within a growth environment, or on specific targeted substrates and mycelium surfaces may vary from programmed mist deposition rates. For the purposes of this disclosure, “Realized Mist Deposition Rates” may be defined as the actual rate of mist deposition (either deposited or not deposited) or actual mist deposition rate at a specific location. After the first few days of a growth cycle, peak metabolic rates are realized, and air cooling may be employed to control for environmental temperature. During this time, mist deposition rates may be programmed to decrease compared to the beginning and end of the growth cycle. Electrostatic misting technology may assist in flattening this mist deposition rate curve and may stabilize the amount of mist being deposited throughout the entire growth cycle of extra-particle aerial mycelium, or a portion thereof, by providing real-time confirmation of Realized Mist Deposition Rates, thereby providing mist when and where it is actually needed to support extra-particle aerial mycelium growth at the appropriate times in the life cycle of a fungal organism growing in a growth environment. Use of such a system may therefore provide efficient use of energy and water resources, thereby reducing usage of input water and nutrients.

### Definitions

The extra-particle aerial mycelia of the present disclosure are growth products obtained from a growth matrix incubated for a period of time (i.e., an incubation time period) in a growth environment, as disclosed herein. The extra-particle aerial mycelium may be separated from a growth matrix, resulting in an aerial mycelium that consists essentially of fungal mycelium and does not include any substrate or growth medium.

“Mycelium” as used herein refers to a connective network of fungal hyphae, with mycelia being the plural form of mycelium.

“Hyphae” as used herein refers to branched filament vegetative cellular structures that are interwoven to form mycelium.

“Substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an extra-particle aerial mycelium of the present disclosure. Suitable substrates are disclosed, for example, in U.S. Patent Application Publication US2020/0239830A1 to O'Brien et al., the entire contents of which are hereby incorporated by reference in their entirety, to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US2020/0239830A1, and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets and combinations thereof. Additional useful substrates for the growth of mycelia are disclosed herein. In some embodiments, the materials are solids, thereby enabling solid state fermentation of one or more fungal inocula.

“Growth medium” or “growth media” as used herein refers to a matrix containing a substrate and an optional further source of nutrition that is the same or different than the substrate, wherein the substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Inoculated substrate” as used herein refers to a substrate that has been inoculated with fungal inoculum. For example, an inoculated substrate can be formed by combining an uninoculated substrate with a fungal inoculum. An inoculated substrate can be formed by combining an uninoculated substrate with a previously inoculated substrate. An inoculated substrate can be formed by combining an inoculated substrate with a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular embodiments, a colonized substrate consists essentially of a substrate and a colonizing fungus in a primary myceliation phase. For many fungal species, asexual sporulation occurs as part of normal vegetative growth, and as such could occur during the colonization process. Accordingly, in some embodiments, a colonized substrate of the present disclosure may also contain asexual spores (e.g., conidia). In some embodiments, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further embodiments, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing mist into the growth environment and/or depositing mist onto colonized substrate and/or any ensuing extra-particle growth. In some embodiments, the use of mist can be adjusted, for example, to desired levels and timing, to affect the topology, morphology, density, and/or volume of the growth. In some embodiments, the mist can be aqueous mist, e.g., mist comprising of H₂O.

Any suitable substrate can be used alone, or optionally combined with a nutrient source, as medium to support mycelial growth. The growth medium can be hydrated to a final target moisture content prior to inoculation with a fungal inoculum. In a non-limiting example, the substrate or growth medium can be hydrated to a final moisture content of at least about 50% (w/w), at most about 95% w/w, within a range of about 50% to about 95%, about 50% to about 90%, about 50% to 85%, about 50% (w/w) to about 80% (w/w), about 50% (w/w) to about 75% (w/w), within a range of about 50% (w/w) to about 65% (w/w), within a range of about 50% (w/w) to about 60% (w/w), or within a range of about 60% (w/w) to about 70% (w/w). Growth medium hydration can be achieved via the addition of any suitable source of moisture. In a non-limiting example, the moisture source can be airborne or non-airborne liquid phase water (or other liquids), a liquid solution containing one or more additives (including but not limited to a nutrient source), and/or gas phase water (or other compound). In some embodiments, at least a portion of the moisture is derived from steam utilized during bioburden reduction of the growth medium. In some embodiments, inoculation of the growth medium with the fungal inoculum can include a further hydration step to achieve a target moisture content, which can be the same or different than the moisture content of the growth media. For example, if growth media loses moisture during fungal inoculation, the fungal inoculated growth media can be hydrated to compensate for the lost moisture.

Methods for the production of extra-particle aerial mycelium disclosed herein can include an inoculation stage, wherein an inoculum is used to transport an organism into a substrate. The inoculum, which carries a desired fungal strain, is produced in sufficient quantities to inoculate a target quantity of substrate. The inoculation can provide a plurality of myceliation sites (nucleation points) distributed throughout the substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and fungi. The inoculum may contain enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary spawn types provided by inoculum retailers. In some embodiments, a fungal inoculum can be characterized by its density. In some embodiments, a fungal inoculum has a density of about 0.1 gram per cubic inch to about 10 grams per cubic inch, or from about 1 gram per cubic inch to about 7 grams per cubic inch. A skilled person can modify variables including the substrate or growth media component identities, substrate or growth media nutrition profile, substrate or growth media moisture content, substrate or growth media bioburden, inoculation rate, and inoculum constituent concentrations to arrive at a suitable medium to support aerial mycelial growth. In some embodiments, the inoculation rate can be expressed as a percentage of the target volume of the substrate or growth media (% (v/v)). In some embodiments, the inoculation rate can range from about 0.1% (v/v) to about 80% (v/v). In some embodiments, the inoculation rate is at most about 50% (v/v), at most about 45% (v/v), at most about 40% (v/v), at most about 30% (v/v), at most about 25% (v/v), at most about 20% (v/v), at most about 15% (v/v), at most about 10% (v/v) or at most about 5% (v/v). In some embodiments, the inoculation rate is about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 18% (v/v), about 19% (v/v), about 20% (v/v), about 21% (v/v), about 22% (v/v), about 23% (v/v), about 24% (v/v), about 25% (v/v), about 26% (v/v), about 27% (v/v), about 28% (v/v), about 29% (v/v) or about 30% (v/v); or any range therebetween. In some embodiments, the inoculation rate can be expressed as a percentage of the target dry mass of the substrate or growth media (% (w/w)). In some embodiments, the inoculation rate can range from about 0.1% (w/w) to about 80% (w/w). In some embodiments, the inoculation rate is at most about 50% (w/w), at most about 45% (w/w), at most about 40% (w/w), at most about 30% (w/w), at most about 25% (w/w), at most about 20% (w/w), at most about 15% (w/w), at most about 10% (w/w) or at most about 5% (w/w). In some embodiments, the inoculation rate is about 1% (w/w), about 2% (w/w), about 3% (w/), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w) or about 30% (w/w); or any range therebetween.

“Aerial mycelium” as used herein refers to mycelium obtained from extra-particle aerial mycelial growth, and which consists essentially of fungal mycelium and does not include any substrate or growth medium.

“Extra-particle aerial mycelium” as used herein refers to mycelium whose aerial mycelial growth occurs away from and outward from the surface of a growth matrix (hence “extra-particle”). In some embodiments, the extra-particle aerial mycelium includes aerial hyphae of a mycelium growing in and/or on a colonized substrate. For example, a growing mycelium in and/or on a colonized substrate can produce hyphae that emerge from and proliferate independently of direct contact with the growth substrate, thereby producing a portion of the mycelium that does not include substrate or growth matrix. In some embodiments, extra-particle aerial mycelium can exhibit negative gravitropism. In a geometrically unrestricted scenario, extra-particle aerial mycelium growth can be positively, negatively or neutrally gravitropic. In some embodiments, extra-particle aerial mycelium growth can be radial, wherein hyphal growth expands in all directions from a point of inoculation and/or germination of, e.g., a growth matrix. In some embodiments, external inputs, such as airflow, can be applied to the extra-particle aerial mycelium as it grows, thereby affecting the direction of growth of hyphae. For example, downward airflow can be applied to extra-particle aerial mycelium growth in the direction of gravity. Alternatively, airflow can be applied across the growth matrix in a manner parallel or horizontal to the growth matrix surface (which contains substrate).

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit negative gravitropism. Without being bound by any particular theory, this may be attributable at least in part to the geometric restriction of the growth format (e.g., the position of a support structure holding the growth matrix (including the particulate substrate), wherein an uncovered tool having a bottom and side walls contains a growth matrix. With such geometric restriction, growth will primarily occur along the unrestricted dimension(s), which in the scenario is primarily vertically (negatively gravitropic) if the tool is positioned such that its opening is facing vertically upward in orientation.

Extra-particle aerial mycelia of the present disclosure can be grown in a matter of weeks or days. This feature is of practical value in the production of food ingredients or food products, where time and efficiency are at a premium. Accordingly, the presently disclosed method of making an extra-particle aerial mycelium comprises incubating a growth matrix in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 days to about 13 days, or within a range of about 7 days to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

Advantageously, incubating a growth matrix comprising a colonized substrate (wherein said colonized substrate comprises a growth medium previously colonized with mycelium of a fungus) in a growth environment of the present disclosure can result in earlier expression of extra-particle aerial mycelial tissue compared to incubation of a growth matrix comprising substantially the same or a similar growth medium and a fungal inoculum, wherein the fungal inoculum contains a fungus. Accordingly, a method of making an extra-particle aerial mycelium of the present disclosure can comprise incubating a growth matrix comprising a colonized substrate (wherein said colonized substrate comprises a growth medium previously colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, extra-particle aerial mycelia of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., extra-particle aerial mycelial growth of a predetermined thickness) without the formation of visible fruiting bodies.

“Dry mass (DM) yield” as used herein refers to the bone-dry mass yield of aerial mycelium from a standard mass of solid substrate. This is representative of the bioefficiency of the organism in converting the solid-substrate components into harvestable aerial mycelium.

### Definitions and Methods Related to Growth Environment

U.S. Patent Application Publication 2015/0033620 to Greetham et al., the entire contents of which is hereby incorporated by reference in its entirety to the extent not inconsistent with the content of this disclosure, describes techniques for growing a material comprising aerial mycelium, referred to in that application as a “mycological biopolymer.” As described therein, a mycological biopolymer product provided by the disclosed method is characterized as containing a homogenous biopolymer matrix that is comprised predominantly of fungal chitin and trace residues (e.g., beta-glucan, proteins). The mycological biopolymer is up-cycled from domestic agricultural lignocellulosic waste and is made by inoculating the substrate made of domestic agricultural lignocellulosic waste with a selected fungus in a container that is sealed off from the ambient environment external to the container. In addition to the substrate and fungal inoculum, the container contains a void space. A network of undifferentiated aerial mycelium comprising a chitin-polymer grows into and fills the void space of the container. The chitin-polymer-based aerial mycelium is subsequently extracted from the substrate and dried. As further described in US2015/0033620, the environmental conditions for producing the mycological biopolymer product described therein, i.e., a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

In some embodiments, the present disclosure provides an extra-particle aerial mycelium grown using the described methods and apparatus. In some further embodiments, the extra-particle aerial mycelium does not contain a visible fruiting body.

As described in International Patent Publication WO2019/099474A1 to Kaplan-Bie et al., the entire contents of which is hereby incorporated by reference in its entirety to the extent not inconsistent with the content of this disclosure, another method of growing a mycological biopolymer material employs incubation of a substrate with nutritive value inoculated with a fungus in containers that are placed in a closed growth environment with air flows passed over each container while the chamber is maintained with a predetermined environment of humidity, temperature, carbon dioxide, and oxygen.

The extra-particle aerial mycelia of the present disclosure are growth products obtained from an inoculated substrate incubated for a period of time (i.e., an incubation time period) in a growth environment, as disclosed herein.

In some embodiments, a method of growing/making an extra-particle aerial mycelium (ultimately an aerial mycelium) of the present disclosure comprises placing a growth matrix in contact with a tool or support structure in the described growth environment. In some embodiments, the tool can comprise a base comprising a surface area. In some embodiments, the surface area can be at least about 1 square inch. In some embodiments, the surface area can be at most about 2,000 square feet. In some embodiments, the growth matrix can be placed in contact with the base, e.g., placed on top of or distributed across the base. In some embodiments, the base can be a planar surface. Non-limiting examples of a tool or support structure include a tray, a sheet, a table, a shelf, a rack, or a conveyer belt. In some embodiments, the tool can have at least one vertical wall. In some embodiments, the base and the at least one vertical wall can together form a cavity. In some embodiments, the growth matrix can be placed or packed in the tool cavity. In some embodiments, the tool can be an uncovered tool. In some other embodiments, the tool can have a lid, the lid having at least one opening, or the tool can be covered at least in part with a perforated barrier. Non-limiting embodiments of a tool having a lid with an opening are disclosed in US2015/0033620A1 to Greetham et al. An uncovered tool, or a tool having a lid with an opening or a perforated barrier, and further having growth matrix on or within the tool, can allow for mist to be deposited onto the growth matrix surface, and/or onto any resulting mycelial growth.

“Growth environment” as used herein refers to an environment that supports the growth of mycelia, as would be readily understood by a person of ordinary skill in the art in the mycelial cultivation industry, and which contains a growth atmosphere having a gaseous environment of carbon dioxide (CO₂), oxygen (O₂), and a balance of other atmospheric gases including nitrogen (N₂), and is further characterized as having a relative humidity. In some aspects of the present disclosure, the growth atmosphere can have a CO₂ content of at least about 0.02% (v/v), at least about 5% (v/v), less than about 8% (v/v), less than about 10% (v/v), between about 0.02% and 10%, between about 0.02% and 8%, between about 5% and 10%, or between about 5% and 8%. In some other embodiments, the growth atmosphere can have an O₂ content of at least about 12% (v/v), or at least about 14% (v/v), and at most about 21% (v/v). In yet other aspects, the growth atmosphere can have an N₂ content of at most about 79% (v/v). Each foregoing CO₂, O₂, or N₂ content is based on a dry gaseous environment, notwithstanding the growth environment atmosphere relative humidity.

In some further embodiments, a method of making an extra-particle aerial mycelium of the present disclosure can comprises incubating the growth matrix in a growth environment, wherein the growth environment has a temperature that supports mycelial growth. In some embodiments, the growth environment has a temperature within a range of about 55° F. to about 100° F., or within a range of about 60° F. to about 95° F. In some more particular embodiments, the growth environment has a temperature within a range of about 80° F. to about 95° F., or within a range of about 85° F. to about 90° F. throughout the incubation time period. In other embodiments, the growth environment has a temperature within a range of about 60° F. to about 75° F., within a range of about 65° F. to about 75° F., or within a range of about 65° F. to about 70° F. In some embodiments, the growth environment temperature can be tuned to optimize for the growth of a particular fungal genus, species, or strain.

In some embodiments, the growth environment suitable for the growth of the extra-particle aerial mycelia of the present disclosure can be a dark environment. “Dark environment” as used herein in connection with a growth environment would be readily understood by a person of ordinary skill in the art in the mycelial cultivation industry and refers to an environment without natural or ambient light, and without growing lights.

Exposing fungi to white light, and especially blue light, has been associated with the induction of fruiting and the enhancement of production efficiency of oyster mushrooms (e.g., see I. Roshita, S. Y. Goh; Effect of exposure to different colors light emitting diode on the yield and physical properties of grey and white oyster mushrooms. AIP Conf. Proc. 9 Nov. 2018; 2030 (1): 020110. https://doi.org/10.1063/1.5066751), the entire contents of which are hereby incorporated by reference in their entirety to the extent not inconsistent with the content of this disclosure. Surprisingly, Applicant has discovered that an extra-particle aerial mycelium for some genera of the present disclosure absent visible fruiting bodies, can be prepared by the methods of the present disclosure in the presence of white light, which includes blue light. Extra-particle aerial mycelium prepared in the presence of white light was consistent in yield, thickness, density, morphology and in the absence of visible fruiting bodies when compared to control aerial mycelia produced under the same growth conditions but in a dark environment. Thus, in some embodiments, a growth environment suitable for the growth of the extra-particle aerial mycelia of the present disclosure is not a dark environment. In some embodiments, the growth environment does not exclude light. In some embodiments, the growth environment can include natural light. In some embodiments, the growth environment can include ambient light. In some embodiments, the growth environment can include a growing light.

As disclosed in US2015/0033620 to Greetham et al., environmental conditions for producing a mycological biopolymer include a CO₂ content of about 3% to about 7% (v/v) to prevent full differentiation of the fungus into a mushroom. Accordingly, in some embodiments, the present disclosure provides for methods of producing an extra-particle aerial mycelium in a growth environment comprising a growth atmosphere, wherein the growth atmosphere can have a CO₂ content within a range of about 3% (v/v) to about 7% (v/v), or within a range of about 5% (v/v) to about 7% (v/v). In some embodiments, the growth atmosphere can have a CO₂ content of about 3%, about 4%, about 5%, about 6%, or about 7% (v/v), or any range therebetween.

Extra-particle aerial mycelium of the present disclosure can be produced without visible fruiting bodies under conditions wherein aqueous mist is introduced into a growth environment having a growth atmosphere containing much lower CO₂ content. For example, it has been found that extra-particle aerial mycelia obtained from a growth environment of circulating mist and an atmosphere having a mean CO₂ content of about 0.04% (v/v) over the course of the incubation time period or having a mean CO₂ content of about 2% (v/v) over the incubation time period were similar in yield, thickness, density, and morphology to extra-particle aerial mycelia obtained via growth in an atmosphere having a mean CO₂ content of 5% (v/v) but otherwise identical growth conditions. Furthermore, extra-particle aerial mycelia of increased thickness can be obtained via incubation in a growth environment described herein and characterized as having particular misting profiles. The present disclosure advantageously provides for methods of making extra-particle aerial mycelia of increased thickness, absent visible fruiting bodies, by adopting preselected misting profiles and employing misting deposition methodologies, without requiring a high CO₂ content growth environment. The ability to increase extra-particle aerial mycelial thickness, absent visible fruiting bodies, by tuning mist deposition uniformity and rate can also advantageously reduce incubation time periods, thereby allowing more efficient production of extra-particle aerial mycelia and reduced risk of microbial contamination that can occur in high moisture environments.

Thus, the present disclosure provides methods of growing extra-particle aerial mycelia (and ultimately aerial mycelium) in a growth environment comprising a growth atmosphere having markedly reduced CO₂ content than found in nature, and with uniform and/or controlled deposition of mist. Accordingly, in some embodiments, the growth atmosphere CO₂ content can be less than about 3% (v/v). In some embodiments, the growth atmosphere CO₂ content can be no greater than about 2.9% (v/v), no greater than about 2.8% (v/v), no greater than about 2.7% (v/v), no greater than about 2.6% (v/v) or no greater than about 2.5% (v/v). In some further embodiments, the growth atmosphere CO₂ content can be less than 2.5% (v/v). In some embodiments, a growth atmosphere of the present disclosure can have a CO₂ content of at least about 0.02% (v/v). In some embodiments, a growth atmosphere of the present disclosure can have a CO₂ content of at least about 0.03% (v/v). In some further embodiments, the growth atmosphere CO₂ content can approximate ambient atmospheric CO₂ content; for example, the growth atmosphere CO₂ content can be at least about 0.04% (v/v). In some more particular embodiments, the growth atmosphere CO₂ content can be within a range of about 0.02% to about 3% (v/v), about 0.02% to about 2.5% (v/v), about 0.03% to about 3% (v/v), about 0.03% to about 2.5% (v/v), about 0.04% to about 3% (v/v), or about 0.04% to about 2.5% (v/v).

In other embodiments, the growth atmosphere CO₂ content can be within a wider range. Thus, in some embodiments, the growth atmosphere CO₂ content can be within a range of about 0.02% to about 7% (v/v), within a range of about 0.04% to about 7% (v/v), within a range of about 0.1% to about 7% (v/v), within a range of about 0.2% to about 7% (v/v), within a range of about 1% to about 7% (v/v), or within a range of about 2% to about 7% (v/v); or can be within a range of about 0.02% to about 5% (v/v), within a range of about 0.04% to about 5% (v/v), within a range of about 0.1% to about 5% (v/v), within a range of about 0.2% to about 5% (v/v), or within a range of about 1% to about 5% (v/v). In some more particular embodiments, the growth atmosphere CO₂ content can be about 1%, about 2%, about 3%, or any range therebetween. In yet other embodiments, the growth atmosphere CO₂ content can be a mean CO₂ content over the course of the incubation time period. In some embodiments, the growth atmosphere mean CO₂ content can be less than about 3% (v/v), less than 2.5% (v/v), or no greater than about 2% (v/v) over the course of the incubation time period.

It is understood that fungal growth requires respiration, which can increase CO₂ content and decrease oxygen (O₂) content in the growth atmosphere, particularly in an enclosed or substantially enclosed growth environment. In some aspects, the present disclosure provides for a growth environment having a growth atmosphere that is maintained during the incubation time period by replenishing the growth environment with one or more of the atmospheric gases, such as CO₂, replenishing the growth environment with air having the same composition as the target growth atmosphere composition, venting the growth environment to reduce content of one or more gases, or a combination thereof. In a non-limiting example, if the CO₂ content in a growth environment is below a target set point, CO₂ gas can be infused into the growth environment. Conversely, if the CO₂ content exceeds a target set point, then fresh air having the target growth atmosphere composition can be introduced into the growth environment while venting the growth environment to release the existing air having the high CO₂ content. Accordingly, growth environment atmospheric content can be maintained via CO₂ and fresh air infusion to maintain a target CO₂ set point; as such, O₂ and other atmospheric components are maintained indirectly and fluctuate as a function of fungal respiration. In some other embodiments, the present disclosure provides for a growth environment wherein the growth atmosphere CO₂ and O₂ contents are allowed to modulate with fungal respiration without adjusting the growth atmosphere to maintain preselected CO₂ or O₂ content. Thus, the growth environment can be a closed system. The present disclosure also provides for a growth environment wherein the growth atmosphere CO₂ and O₂ contents are allowed to modulate with fungal respiration, and for adjustments to be made to the growth atmosphere under conditions where a particular preselected growth atmospheric condition is exceeded or underperformed by the growth environment. In a non-limiting example, an extra-particle aerial mycelium can be grown in a growth atmosphere that allows for natural fungal respiration to occur, with a preselected CO₂ content ranging from about 0.02% to about 7% CO₂ (v/v), wherein the CO₂ content is adjusted (e.g., by injection of CO₂ into the growth atmosphere) if the CO₂ content falls outside the scope of the preselected range.

A growth environment of the present disclosure can be further characterized as having an atmosphere a pressure as would be readily understood by a person of ordinary skill in the art in the mycelial cultivation industry. In a non-limiting embodiment, a growth atmosphere of the present disclosure can have an atmospheric pressure within a range of about 27 to about 31 inches of mercury (Hg), can have an atmospheric pressure of about 29 to about 31 inches Hg, or can have an atmospheric pressure of about 29.9 inches Hg. In some embodiments, a growth environment of the present disclosure can be characterized as having an ambient atmospheric pressure.

In some aspects of the present disclosure, the growth environment suitable for the growth of the extra-particle aerial mycelia of the present disclosure is characterized as having an airflow. In some further embodiments, the air composition of the airflow can be substantially the same as the composition of the growth environment atmosphere. In some embodiments, an airflow can be used to direct and/or deposit mist that is present in the growth environment towards or onto a growth matrix. The skilled person can adopt various means of directing the flows of air, including baffles, perforated barriers, airflow boxes and/or other tools that can be suitably positioned in the growth environment or in relation to tools or beds containing growth matrix in order to achieve the desired outcome, including a homogeneous airflow, with respect to direction and/or velocity, across a plurality of growth matrices in the growth environment, and/or a homogeneous introduction and/or deposition of mist in the growth environment. However, even with such structural airflow intervention techniques, sometimes the vast size of the growth environment (e.g., commercial scale) and/or hinderances by physical structures contained within the growth environment, may continue to pose challenges with efforts to completely homogenize airflow, or introduction and/or deposition of mist. Therefore, other homogenizing techniques as described herein, provide increased opportunity for achieving desired airflow and mist deposition objectives.

“Horizontal airflow” as used herein refers to flows of air directed substantially parallel to the surface of a growth matrix and any subsequent extra-particle mycelial growth.

In some other embodiments the method of preparing an extra-particle aerial mycelium of the present disclosure can include directing an airflow through the growth environment. In some embodiments, the airflow can be a relatively high airflow environment, wherein the airflow can have a velocity of greater than about 250 linear feet per minute (lfm). In other embodiments, the airflow can be a relatively lower airflow environment, wherein the airflow can have a velocity of less than about 150 lfm, less than about 125 lfm, less than about 100 lfm or less than about 75 lfm. In some more particular embodiments, the growth environment can have an airflow, wherein the airflow velocity is less than about 50 lfm, less than about 40 lfm, less than about 30 lfm or less than about 25 lfm.

In some embodiments, the airflow is a substantially horizontal airflow. In some embodiments, the substantially horizontal air flow can have a velocity of no greater than about 350 lfm, or a velocity no greater than about 300 lfm. In other embodiments, the substantially horizontal airflow can have a velocity of no greater than about 275 lfm, a velocity of no greater than about 175 lfm, a velocity of no greater than about 150 lfm, a velocity of no greater than about 125 lfm, or a velocity of no greater than about 110 lfm. In some further embodiments, the velocity is at least about 5 lfm, at least about 10 lfm, at least about 15 lfm, at least about 20 lfm, at least about 25 lfm, at least about 30 lfm, at least about 35 lfm, at least about 40 lfm, at least about 45 lfm or at least about 50 lfm. In some more particular embodiments, the substantially horizontal airflow has mean velocity of about 5 lfm, about 10 lfm, about 15 lfm, about 20 lfm, about 25 lfm, about 30 lfm, about 35 lfm, about 40 lfm, about 45 lfm, about 50 lfm, about 55 lfm, about 60 lfm, about 65 lfm, about 70 lfm, about 75 lfm, about 80 lfm, about 85 lfm, about 90 lfm, about 95 lfm, about 100 lfm, about 105 lfm, about 110 lfm, about 115 lfm or about 120 lfm. In some more particular embodiments still, the substantially horizontal air flow can have a velocity within a range of about 5 lfm to about 125 lfm, within a range of about 5 lfm to about 100 lfm, within a range of about 5 lfm to about 75 lfm, or within a range of about 5 lfm to about 50 lfm. In yet more particular embodiments, the substantially horizontal air flow can have a velocity within a range of about 5 lfm to about 40 lfm, or within a range of about 5 to about 25 lfm. In other embodiments, the substantially horizontal air flow can have a velocity within a range of about 40 lfm to about 120 lfm. Without being bound to any particular theory, the flows of air can facilitate the distribution of mist throughout the growth environment, can facilitate the distribution of mist onto the growth matrix surface and/or extra-particle mycelial growth, or both. The air flow and misting methods and apparatus, including the complimentary electrostatic charging mist methods and apparatus described herein, can be tuned in concert to achieve the desired mist deposition rate and/or mean mist deposition rate, and to tune the mycelial tissue morphology. It would be expected that the electrostatic charging mist methods (and accompanying apparatus and systems thereof), could also provide airborne mist particle separation, dispersion, and distribution independent of certain described airflow interventions.

“Mist” as used herein refers to a fine spray or dispersion of liquid droplets suspended in air or another gas. In some embodiments, the mist can comprise liquid water (including liquid water comprising one or more solutes, ions, electrolytes, non-electrolytes, moieties, insoluble constituents, etc.) and/or any other suitable liquid for growing extra-particle aerial mycelium. In some embodiments, the air can comprise air within a growth environment.

“Mist deposition rate” as used herein refers to the rate at which mist is deposited per discrete instance of mist deposition. Any standalone usage herein of “mist deposition rate,” without the prefix “mean,” refers to the rate at which mist is deposited per discrete instance of mist deposition and is used interchangeably herein with “instantaneous mist deposition rate” or “momentary mist deposition rate.” “Mean mist deposition rate” is not used interchangeably herein with respect to “mist deposition rate” and is as defined elsewhere herein. The mist deposition rate can be based on or determined by measuring the volume of mist deposited on a surface area over a period of time, wherein the period of time is a fraction of the total incubation time period. In a non-limiting example, the mist is deposited on an exposed surface of growth matrix at a mist deposition rate of about 1 microliter per square centimeter of growth matrix per hour. In another non-limiting example, the mist is deposited on extra-particle aerial mycelial growth, and the mist deposition rate is about 1 microliter per square centimeter of the extra-particle aerial mycelial growth per hour. In some embodiments, the mist deposition rate can be reported as the volume of mist deposited per misting duty cycle. For the purposes of the present disclosure, a mist deposition rate of 1 microliter per centimeter squared per hour (1 μL/cm²/hour) is substantially equivalent to a mist deposition rate of 1 milligram per centimeter squared per hour (1 mg/cm²/hour), solute concentration notwithstanding.

In some embodiments, the mist can be continuously introduced into the growth environment. In some further embodiments, the continuous introduction of mist can be pulse-width modulated (i.e., controlling mist deposition by varying misting apparatus duty cycle and periodicity). In some other embodiments, the continuous introduction of mist deposition can occur at a fixed rate. In yet some other embodiments, the continuous introduction of mist deposition can occur at a variable rate.

In other embodiments, the mist can be intermittently introduced into the growth environment. In some further embodiments, the intermittent introduction of mist can occur at a fixed rate. In other further embodiments, the intermittent introduction of mist can occur at a variable rate. In other further embodiments, the intermittent introduction of mist can occur at regular or irregular periods. In other further embodiments, the intermittent introduction of mist can occur with regular or irregular intervals therebetween without mist introduction.

In some embodiments, a misting apparatus can be operated at a particular duty cycle, a duty cycle defining a percentage of time that the misting apparatus is producing mist. In some embodiments, the misting apparatus is operated at a duty cycle of about 100%. In some embodiments, the misting apparatus is operated at a duty cycle within a range of about 0.1% to about 100%. In some embodiments, the misting apparatus is operated at a duty cycle within a range of about 1% to about 100%, about 5% to about 100%, about 10% to about 100%, about 15% to about 100%, about 20% to about 100% or about 25% to about 100%. In some other embodiments, the misting apparatus is operated at a duty cycle of less than 100%. In some embodiments, the misting apparatus is operated at a duty cycle of no greater than about 75%, no greater than about 50%, no greater than about 40%, no greater than about 30%, no greater than about 25%, no greater than about 20% or no greater than about 15%. In some further embodiments, the misting apparatus is operated at a duty cycle of at least about 1%, at least about 5%, at least about 10%, at least about 15%, at least about 20% or at least about 25%. In some more particular embodiments, the misting apparatus is operated within a range of about 1% to about 15%, about 5% to about 25%, about 25% to about 50%, about 50% to about 75%, or about 75% to about 100%.

In some embodiments, a duty cycle can be further characterized by a cycle period. Non-limiting examples include a duty cycle period of about 3600 second (i.e., about 1 hour), about 1800 seconds (i.e., about 30 minutes), about 360 seconds, (i.e., about 6 minutes), about 180 seconds (i.e., about 3 minutes), about 60 seconds (i.e., about 1 minute), about 45 seconds, about 30 seconds, about 15 seconds, or any value or range therebetween. In some embodiments, the duty cycle period may be between about 15 seconds to about 30 seconds, alternatively between about 30 seconds and about 45 seconds, alternatively between about 45 seconds and about 60 seconds, or alternatively between about 36 seconds and 45 seconds. In some embodiments, a duty cycle period can be at most about 60 minutes, at most about 30 minutes, at most about 15 minutes, or at most about 10 minutes. In some other embodiments, a duty cycle period can be at most about 9 minutes, at most about 8 minutes, at most about 7 minutes or at most about 6 minutes.

As disclosed herein, a method of making an aerial mycelium of the present disclosure can include introducing mist into the growth environment throughout an incubation time period. Introducing mist “throughout the incubation time period” as used herein refers to introducing the mist from the beginning of the incubation time period to the end of the incubation time period. In some embodiments, introducing mist into the growth environment can comprise operating a misting apparatus at a duty cycle of greater than zero from the beginning of the incubation time period to the end of the incubation time period. In a non-limiting example, introducing mist into a growth environment throughout the incubation time period can comprise operating a misting apparatus at a 50% duty cycle from the beginning of the incubation time period to the end of the incubation time period. Further to this non-limiting example, the misting apparatus operating at the 50% duty cycle can have a duty cycle period of at most about 10 minutes. Thus, in this non-limiting example, the misting apparatus can operate (and thus release mist) for 5 minutes out of each 10-minute duty cycle period, and each 10-minute duty cycle period repeats from the beginning of the incubation time period to the end of the incubation time period. Similarly, introducing mist “throughout a portion of the incubation time period” as used herein refers to introducing the mist from the beginning of the portion of the incubation time period to the end of the portion of the incubation time period. In some embodiments, the end of the portion of the incubation time period can be the end of the entire incubation time period. In some embodiments, introducing mist into the growth environment throughout a portion of the incubation time period can comprise operating a misting apparatus at a duty cycle of greater than zero from the beginning of the portion of the incubation time period to the end of the portion of the incubation time period. It will be understood that introducing mist “throughout the incubation time period” and “throughout a portion of the incubation time period” as used herein can include, but do not require, mist introduction at exactly the beginning of, nor exactly the end of the incubation time period or the portion of the incubation time period, for example, in embodiments where the mist is not applied continuously throughout the entirety of the incubation time period or the portion of the incubation time period.

“Mean mist deposition rate” as used herein refers to a mist deposition rate averaged over an incubation time period. The mean mist deposition rate can be expressed based on a surface area over which the mist is deposited. In a non-limiting example, the mist is deposited on an exposed surface of growth matrix at a mean mist deposition rate of about a microliter per square centimeter of growth matrix per hour. In another non-limiting example, the mist is deposited on an exposed surface of growth matrix containing extra-particle aerial mycelial growth, and the mean mist deposition rate is about 1 microliter per square centimeter of the growth matrix containing the extra-particle aerial mycelial growth per hour. For the purposes of the present disclosure, a mean mist deposition rate of 1 microliter per centimeter squared per hour (1 μL/cm²/hour) is substantially equivalent to a mean mist deposition rate of 1 milligram per centimeter squared per hour (1 mg/cm²/hour), solute concentration notwithstanding.

“Electrostatic mist” or “Electrostatically charged mist” as used herein, refers to a fine particle of mist (e.g., of water) with an electrostatic charge that has been imparted via a variety of methods including but not limited to induction, electrified misting nozzles, and/or high velocity air fans. It should be understood that such fine particle of mist (e.g., of water) may include additives to provide additional benefits to growing mycelium, such as nutritional additives, or alternatively, nutritionally neutral materials (materials that offer no nutritional benefits, but which also do not present any deleterious effects to growing mycelium), but which assist in holding charges in the water particles (as may be advantageous in high humidity environments) or for serving in another capacity, e.g., contamination control and/or adding other uniform properties to growing extra-particle aerial mycelium.

In some embodiments, it is expected that the misting apparatus efficiency and airflow/misting efficacy to various zones within the growth environment can be both effectuated and validated using electrostatically charged mist droplets. Use of such electrostatically charged mist droplets can assist in the determination of either the presence or absence of mist deposition at specific locations within a growth environment, or alternatively, quantitative information regarding mist deposition in a particular growth environment location. For instance, it is expected that strength or density of charge in particular areas may be calibrated to mist quantities and assist in forming a determination of mist deposition quantities in those locations. In further embodiments, by monitoring the charges present at specific locations, a mist apparatus may be triggered to increase or decrease mist disbursement through an airflow system to one or more locations within a growth environment. Therefore, in further alternative embodiments, electrostatically charged mist particles may be used to selectively place mist at distinct locations within a growth environment. Alternatively, electrostatically charged mist may facilitate a more uniform dispersal of mist within a growth environment and across a growth matrix (or substrate), through either charge attraction (e.g., attraction to an oppositely charged substrate) or repulsion (e.g. from a similarly charged substrate, or alternatively, between various similarly charged mist particles in the growth environment atmosphere).

In some embodiments, the method of making an extra-particle aerial mycelium of the present disclosure can comprise introducing electrostatically charged mist into the growth environment via a misting apparatus throughout the incubation time period, or a portion thereof. In certain embodiments, the introduction of the mist may be controlled through electrostatic charge sensing apparatus, such that the mist may be more appropriately dispersed throughout the growth chamber during the incubation time period, or portion thereof, or fungal life cycle, or portion thereof (when it is needed by the specific fungal organism to grow (such as based on respiration monitoring equipment).

In some embodiments, extra-particle aerial mycelia can be prepared by exposing a growth matrix to mist throughout a portion of the incubation time period (e.g., by introducing mist into the growth environment throughout a portion of the incubation time period). For example, a portion of the incubation time period can include a period of time that is less than or equal to the incubation time period. Applicant has measured vertical expansion kinetics of mycelia over the course of an entire incubation period and has characterized the kinetics as having a primary myceliation phase and a vertical expansion phase. The primary myceliation phase included days 1 to 3 of the incubation time period. Introducing mist throughout a portion of the incubation time period (wherein the portion included the vertical expansion phase), and not introducing mist on days 1 to 3 of the incubation time period was sufficient to produce extra-particle aerial mycelium having substantially similar characteristics to aerial mycelia obtained by depositing mist throughout the entire incubation period. In one embodiment, the Realized Mist Deposition Rate determined by electrostatic charge monitoring in accordance with the disclosure, can be used to enhance the efficacy of the mist deposition and airflow apparatus at various stages of the incubation time period.

The desired airborne mist concentration value, and/or the control of the airborne mist concentration level in response to the mist concentration value, for improved growth may be different during different phases of the growing cycle (including zero). Further, the desired airborne mist concentration value, and/or the control of the airborne mist concentration level in response to the mist concentration value, for improved growth may also be different based on the organism generating the extra-particle aerial mycelia. Some aspects of the present disclosure provide for a method of growing an extra-particle aerial mycelium comprising exposing a growth matrix to a growth environment comprising mist throughout the incubation time period (e.g., by introducing mist into the growth environment throughout the incubation time period, i.e., throughout the entire incubation time period). In other aspects, the present disclosure provides for a method of making an extra-particle aerial mycelium comprising exposing a growth matrix to aqueous mist throughout a portion of the incubation time period (e.g., by introducing mist into the growth environment throughout a portion of the incubation time period). In some embodiments, a portion of the incubation time period can comprise a vertical expansion phase. In some further embodiments, a portion of the incubation time period can further comprise at least a portion of a primary myceliation phase. In some other embodiments, a portion of the incubation time period can exclude a primary myceliation phase. In yet some other embodiments, a portion of the incubation time period can comprise a vertical expansion phase. Accordingly, in some embodiments, introducing mist into a growth environment throughout a portion of an incubation time period can comprise introducing mist into the growth environment throughout a vertical expansion phase. In some embodiments, introducing mist into the growth environment throughout a portion of the incubation time period can comprise introducing mist into the growth environment throughout a vertical expansion phase and can exclude introducing mist during the primary myceliation phase. In some embodiments, the portion of the incubation time period can terminate at the end of a vertical expansion phase or can terminate at the end of an incubation time period. In each of the above and below described misting variations, electrostatic mist detection apparatus and methods may be used to: 1) determine the presence or absence of mist deposition at various locations in the growth environment, 2) to assist in understanding the quantity of mist deposition at various locations in the growth environment, and 3) to trigger an action in response to the measurement of electrostatically charged mist particles in a growth environment (such as the onset of more mist in one or more locations, the slowing of mist in one or more locations, or the discontinuation of mist in its entirety (either by specific location or in the growth environment as a whole)).

In some embodiments, a portion of an incubation time period can begin during a first day, a second day, a third day or a fourth day of the incubation time period. Accordingly, in some embodiments, introducing mist into a growth environment throughout a portion of an incubation time period can comprise introducing mist into the growth environment during a first, a second, a third or a fourth day of the incubation time period. In some embodiments, the portion of the incubation time period can terminate at the end of a vertical expansion phase or can terminate at the end of an incubation time period.

In some embodiments, the mycelial growth response can be affected by the presence of mist in the growth environment, and/or by mist deposition in the growth environment, and/or by mist deposition on the growth matrix. Applicant has shown that extra-particle aerial mycelium growth does not occur in the absence of mist in the growth environment and/or in the absence of mist deposition in the growth environment, and that extra-particle aerial mycelium growth does occur in the presence of mist in the growth environment, and/or in the presence of mist deposition in the growth environment.

In some embodiments, a growth environment can be provided that has an amount of mist present therein. The amount of mist present can be established before or during various actions taken within the growth environment, for example, during incubating a growth matrix. Thus, in some embodiments, a method of growing an extra-particle aerial mycelium of the present disclosure can include exposing a growth matrix to a growth environment that has an amount of mist present therein. In some embodiments, exposing the growth matrix to the growth environment can include introducing mist into the growth environment. In some embodiments, the mist can be introduced into the growth environment resulting in a detectable quantity of deposited mist in the growth environment. In some more particular embodiments, mist can be introduced into the growth environment resulting in a mean mist deposition rate that results in a detectable quantity of deposited mist in the growth environment. For example, mist can be introduced into the growth environment resulting in a mean mist deposition rate that results in a detectable quantity of deposited mist on surfaces of the container or other structure, on the growth matrix, on the extra-particle aerial mycelial growth, and/or on other structures within the growth environment. Methods of detecting deposited mist include visual inspection methods for visibly detectable deposited mist, measuring a quantity of deposited mist based on mass of collected mist or deposited solute, or other reasonable detection methods. A non-limiting example of a method of measuring an amount of deposited mist can be based upon the method of measuring mean mist deposition rate disclosed herein. Thus, in some embodiments, the mist can be introduced into the growth environment resulting in a mean mist deposition rate that does not result in a measurable mass of deposited mist in the growth environment. This can be confirmed after placing one or more open Petri dishes of known surface area in a growth environment during an incubation time period for at least 24 hours and at most about 7 days. Throughout this incubation time period, some amount of mist is present within the growth environment based upon some amount of mist introduction, allowing for the collection of some theoretical amount of deposited mist in the one or more open Petri dishes. The total theoretical mass of collected mist can be determined (to determine the mass of the deposited mist) and divided by the period of time (to determine the mean mist deposition rate based on mass). In embodiments, if the mist present in the growth environment does not result in measurable mist deposition in the growth environment based on mass, the total amount (i.e., mass) of collected mist is negligible, i.e., not measurable within the tolerance of the balance used to determine the mass, but at some small amount above zero.

In some other embodiments, mist can be introduced into the growth environment resulting in a mean mist deposition rate that does not result in a measurable volume of deposited mist in the growth environment. This can be confirmed after placing one or more open Petri dishes of known surface area in a growth environment during an incubation time period for at least 24 hours and at most about 7 days. Throughout this incubation time period, some amount of mist is present within the growth environment based upon some amount of mist introduction, allowing for the collection of some theoretical volume of deposited mist in the one or more open Petri dishes. The total theoretical volume of collected mist can be determined (to determine the volume of the deposited mist) and divided by the period of time (to determine the mean mist deposition rate based on volume). In embodiments wherein the aqueous mist present in the growth environment does not result in measurable mist deposition in the growth environment based on volume, the total amount (i.e., volume) of collected mist is negligible, i.e., not measurable within the tolerance of the volumetric equipment used to determine the volume, but at some small amount above zero.

In yet other embodiments, the mist can be introduced into the growth environment resulting in a mean mist deposition rate that does not result in visible deposited mist in the growth environment. This can be confirmed after placing one or more open Petri dishes in a growth environment during an incubation time period for at least 24 hours and at most about 7 days. Throughout this incubation time period, some amount of mist is present within the growth environment based upon some amount of mist introduction, allowing for the collection of some theoretical amount of deposited mist in the one or more open Petri dishes. During and/or upon completion of the incubation time period, the one or more open Petri dishes can be visually inspected to confirm that no visible amount of mist deposition is present (functionally, when the one or more Petri dishes are dry).

### DISCUSSION OF THE FIGURES

The following discussion presents detailed descriptions of the several embodiments of apparatus, systems, and methods for growing mycelium, for example through introduction of electrostatic mist (and monitoring of such) within a growth environment, as shown in the figures. These embodiments are not intended to be limiting, and modifications, variations, combinations, etc., are possible and within the scope of this disclosure.

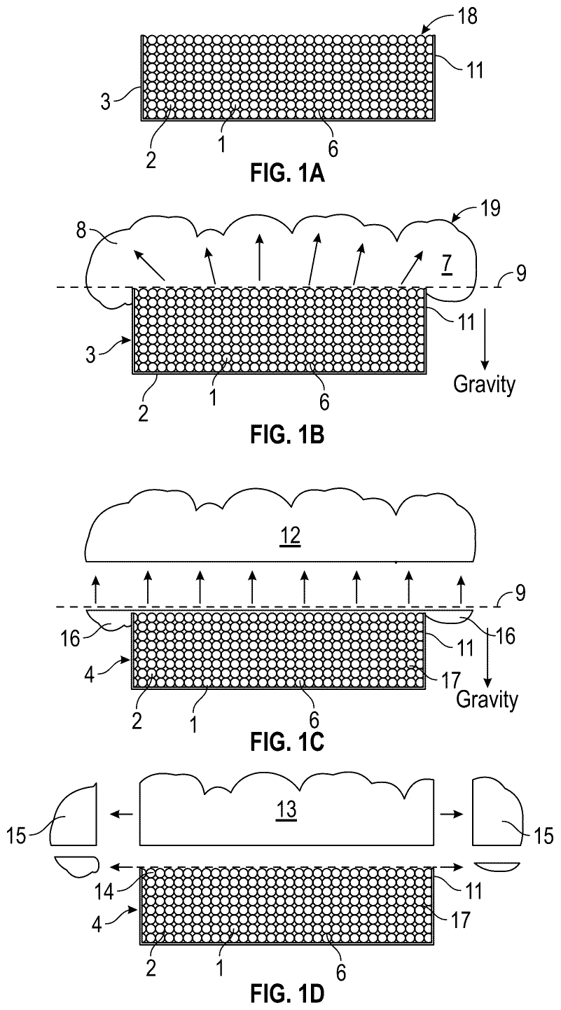

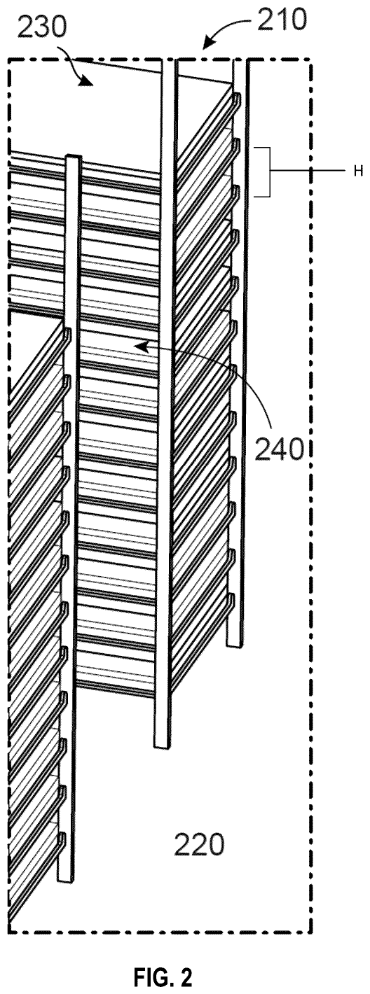

FIG. 1A illustrates an embodiment of a growth matrix 3 suitable to support extra-particle mycelial growth, such as extra-particle aerial mycelial growth. The growth matrix 3 is shown as circles. In some embodiments, the growth matrix 3 can be positioned on (e.g., contained within) a tray (or tool) 11 with side and bottom walls, as shown. The tray, as shown, does not include a lid (but such can be included, if desired). While the tray is shown with the open side facing upward, such configuration is not required, as the tray 11 alternatively may be situated such that the opening is situated facing sideways or downward (in which configuration the growth matrix 3 can be held in place by a textile through which mycelium may grow). The growth matrix 3 can include a growth medium and a fungus. For example, the growth matrix 3 can comprise growth media 2, substrate 1, and colonized (or pre-colonized) substrate 6, to support growth therefrom, or, alternatively, growth media and one or more fungal inocula. The growth matrix 3 can be placed within a growth environment. One or more environmental conditions can be controlled within the growth environment, for example, to affect the growth of mycelium from the growth matrix for desirable extra-particle aerial mycelial growth. For example, a processor with associated sensors, such as condition monitors or thermostats, can be provided to control (via controllers) oxygen (O₂) content, carbon dioxide content (CO₂), temperature, humidity, airflow, misting (and variables related to misting, e.g., electrostatic charge levels, direction, droplet size, etc.) and/or other environmental conditions associated with extra-particle aerial mycelium growth to, from, and/or within the growth environment through an interface. A processor that may or may not be coupled with one or more controller, as disclosed herein, may assist in monitoring and facilitating favorable extra-particle aerial mycelium growth conditions within the growth environment. For example, one or more sensors can be positioned throughout the growth environment such as to monitor one or more growth chamber condition. In some embodiments, the growth matrix 3 can comprise a surface 18.

In some embodiments, the growth matrix 3 is placed in the growth environment without a tray 11 (not shown) For example, the growth matrix can be placed in the growth environment on a planar support structure without side walls, such as a mycological growth web, net, open bed, or open-rail or wire shelf.

FIG. 1B illustrates an embodiment of extra-particle aerial mycelial growth 8 from the growth matrix 3 of FIG. 1A. For example, the growth can occur when the growth matrix 3 from FIG. 1A is incubated or otherwise processed within a growth environment under growth conditions suitable for the desired properties of the extra-particle aerial mycelium growth 8 in FIG. 1B. For example, in some embodiments, the environmental condition(s) within the growth environment can be controlled to induce homogeneous extra-particle aerial mycelial growth from the growth matrix through manipulation of airflow (e.g., control of direction and speed). Such homogeneity of growth may be further induced via the use of electrostatically charged mist depositing apparatus, and optionally accompanied with electrostatically charged mist monitoring apparatus. Therefore in one embodiment, such growth environment can be controlled to induce homogeneous extra-particle aerial mycelial growth from the growth matrix through manipulation of electrostatically-charged mist and airflow. In some embodiments, the substrate, growth media, and/or growth matrix, can be itself charged to increase the homogeneity of the growing extra-particle aerial mycelium. In some further embodiments, the support structure enclosing, holding, or otherwise supporting the substrate, growth media, growth matrix, and/or extra-particle aerial mycelium can be provided a voltage to increase the homogeneity of growing extra-particle aerial mycelium. Homogeneity may, e.g., be the smoothness of the extra-particle aerial mycelial growth, the absence of bulbous formations on the extra-particle aerial mycelial growth surface, or the uniformity of formations upon the extra-particle aerial mycelial growth surface. In some embodiments, an adjacent material or structures within the growth environment (e.g., metallic targets), that is/are adjacent the growth matrix (including substrate or growth media), may also be provided a voltage to create a mist-attracting charge to a location adjacent or proximate to the growth matrix, in order to attract electrostatically charged mist to locations adjacent or proximate to the growth matrix.

The extra-particle aerial mycelium growth can extend away from and outward from a surface of the growth matrix 18 (of FIG. 1A) to form an aerial mycelium 7 as shown, with growth directional arrows provided for further clarity in FIG. 1B. Appropriate growth conditions of the growth matrix 3 in FIG. 1A results in extra-particle aerial mycelium growth initiating across the growth matrix surface 18. Next, extra-particle aerial mycelium growth continues to expand outward forming a volume of extra-particle aerial mycelial growth 8 as shown in FIG. 1B. In some embodiments, the extra-particle aerial mycelium growth 8 can be grown to various heights. In some embodiments, the growth is about 3 to 4 inches high above the growth matrix 3 surface 18. In some embodiments, the growth is considerably higher above the growth matrix, such as at least about 5 inches, or alternatively, at least about 8 inches, or alternatively, at least about 10 inches, or alternatively at least about 12 inches, or alternatively between about 8 to 15 inches, alternatively between about 8 to 12 inches, or alternatively between about 10 to 12 inches, or alternatively, between about 10 to 15 inches above the growth matrix surface 18. Such heights of the extra-particle aerial mycelium can be achieved, for example, in between about two to three weeks of growth, alternatively in about two weeks of growth. It will be understood that although the extra-particle aerial mycelium growth has some amount of irregularity to its upper surface 19 topology as shown in FIG. 1B (i.e., bumpy, or bulbous surface), the drawings are not to scale, and the extra-particle aerial mycelium surface 19 can vary from flat and homogeneous to bumpy and/or bulbous.

In some embodiments, the growth can be implemented on a mycological growth web (alternatively, on a bed, screen, grid-like structure, open rail shelf etc.), for example, without the tray 11 shown. The growth web can include the growth matrix and the extra-particle aerial mycelial growth (e.g., without a tray 11) thereupon. The growth web can include any suitable support structure to support the growth matrix 3 and the extra-particle aerial mycelium growth 8, such as a growing net, screen, or open rail shelf. The web can be a standard size, such as a 63″W×38′L, 63″W×98′L or any of many other web configurations. Other sizes can be implemented, including lengths up to 90, 100 feet, or more. The growing net can comprise one or more layers of a perforated or nonperforated material, or combinations thereof, such as a plastic, nylon (e.g., nylon weave), or any other flexible, suitable material or multiple layers of material for growing extra-particle aerial mycelium growth 8 from a growth matrix 3. The web can extend in length from right to left in the orientation shown in FIG. 1B.

FIG. 1C illustrates an embodiment in which the extra-particle aerial mycelium growth 8 and growth matrix 3 of FIG. 1B have been divided along a separation zone 9 (dot-dashed line) to form a separated aerial mycelium 12 and depleted growth matrix 4 (that is a nutrition-reduced material). Referring also to FIG. 1B, the separation zone 9 (dot-dashed line) can be a zone (e.g., plane) where the extra-particle aerial mycelium growth 8 can be divided and detached from the growth matrix 3. As shown in FIG. 1C, upon detachment, the extra-particle aerial mycelial growth 8 from FIG. 1B has formed a separated aerial mycelium 12, and the growth matrix 3 from FIG. 1B has formed a depleted growth matrix 4 from which at least some nutritional content has been used.

The separation zone 9 can be positioned, and thus the growth matrix 3 and the extra-particle aerial mycelium growth 8 can be divided, such that the depleted growth matrix 4 includes a transitional layer 14 of extra-particle aerial mycelium growth 7 remaining upon the underlying remainder portion 17 of the depleted growth matrix 4 (as seen in FIG. 1D). This transitional layer 14 can prevent any of the growth matrix material 3 from remaining on the separated aerial mycelium 12 after detachment from the extra-particle aerial mycelium growth 8 and can allow for a cleaner, sharper detachment. The inclusion of a transitional layer 14 can be beneficial, for example, in food applications, where the product resulting from the separated aerial mycelium 12 may not be allowed to include any significant amount of growth matrix 3. The separation zone 9 need not be linear as shown, although in some embodiments, it can form a plane extending along the dot-dashed lines shown and approximately perpendicular into the view as shown, to form a plane of separation. For embodiments that implement the tray 11, the division of the extra-particle aerial mycelium growth 8 from the growth matrix may result in portions 16 of the extra-particle aerial mycelium growth 8 that extend below the separation zone 9 to be divided and detached from the separated aerial mycelium 12.

FIG. 1D illustrates an embodiment of the separated aerial mycelium 12 in FIG. 1C that has been cut to form an aerial mycelium panel 13. The cut can be made transversely into the page in the orientation shown. This cut can be across the width of the separated aerial mycelium 12. The portions 15 that are shown cut away from the aerial mycelium panel 13 may be, in some embodiments, part of other aerial mycelium panels 13 that are longitudinally adjacent to panel 13. For example, embodiments without a tray 11 and which implement a mycological web may include adjacent panels 13 rather than portions 15.

The dividing and cutting processes described with reference to FIGS. 1C and 1D, respectively, can be performed with any dividing instrument and cutting instrument, respectively, suitable to divide and/or cut mycelial growth. For example, these instruments can comprise a wire saw, a reciprocating saw (e.g., a reciprocating blade or wire), a recirculating saw (e.g., a recirculating band or wire), any of which can be configured to move in two axes—e.g., across the width of the separated aerial mycelium, and transversely, and/or a single axis “guillotine” cut (e.g., transversely), or combinations thereof. In some embodiments, a rotating cutting instrument, such as a cutting mill or “pizza cutter” configuration, can be implemented. In some embodiments, a double cake slicer blade can be implemented.

FIG. 2 illustrates an embodiment of an isometric view of a system 210 for cultivating extra-particle aerial mycelia within a growth environment 220. The growth environment 220 can be configured to grow extra-particle aerial mycelium from a growth matrix positioned therewithin. For example, system 210 and growth environment 220 can be implemented to facilitate the mycelial growth described above with reference to FIGS. 1A-1B and can include similar components and functionality.

The growth environment 220 can include one or more shelves 240 (e.g., vertically configured shelves on racks), on which a growth matrix can be positioned, and from which extra-particle mycelial growth can extend. The growth matrix can be positioned directly on a shelf, or with an intervening growth support structure, such as a growth web. One or more racks 230, such as the two racks shown (and separated by an aisle), can include a plurality of the shelves 240 (e.g., stacked vertically), positioned within the growth environment 220. The environment can include various numbers of racks, which can include various numbers of shelves 240, and can be of various dimensions. For example, a shelf can be sized and configured to support a web or other growth support structure, such as those described above with reference to FIGS. 1A and 1B, or other growth support structures. In some embodiments, the racks include 12 shelves, and are 12-16 feet high. The shelves 240 can move vertically to compensate for spacing shelves 240 at different heights. The shelves 240 can be spaced apart from each other (e.g., vertically) a spacing height H that is sufficient to allow for the height of the desired extra-particle aerial mycelial growth combined with the height of the underlying growth matrix, sufficient clearance for airflow, mist flow, monitoring, processing, and handling. It will be understood that air, air content, and airflow as used herein can refer to various compositions of various gases, and flow of those gases suitable for the products and methods described herein and should not otherwise be limited to a particular composition or ratio of gases. Additionally, some embodiments herein that are described as being implemented with respect to “water” may be similarly implemented with other liquids.

The spacing height H is defined as the distance between the same two corresponding points on two adjacent shelves 240. For example, the spacing height can be defined as the distance from the top surface of a first lower shelf to the corresponding top surface of an adjacent upper shelf. In some embodiments, the spacing height can be in a range between about 200 mm to about 630 mm, or between about 225 mm to about 490 mm, or between about 250 mm to about 450 mm. In some embodiments, the spacing height can be less than about 630 mm, less than about 490 mm, or less than about 450 mm. In some embodiments, the spacing height can be about 350 mm. These spacing heights can be advantageous because the nature of the mycelial growth herein requires less spacing, and thus can allow for an increased number of shelves and higher output than conventional mushroom cultivation. While shelving units of the types shown may create air flow challenges (and impede currents of air flow, thereby potentially leading to heterogeneous deposition of mist) in a growth environment, use of an electrostatically charged mist system can allow for charged mist particles to repel from one another, thereby overcoming some of the physical air flow challenges created by the rack structural barriers, and increased sized growth environments.

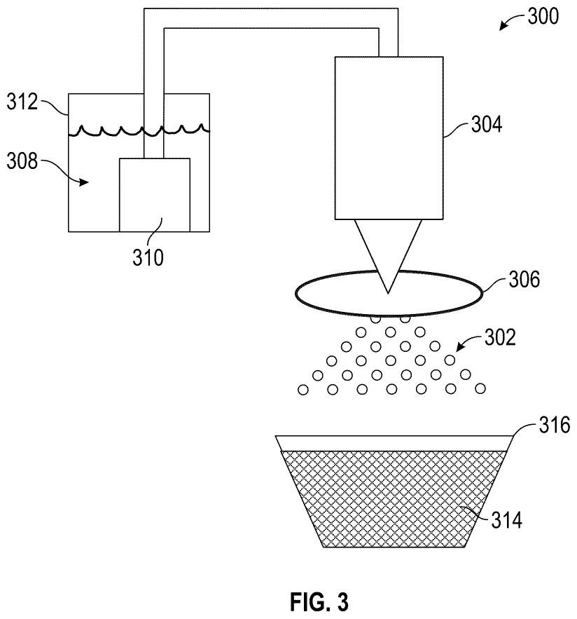

FIG. 3 illustrates an exemplary embodiment of an electrostatically charged misting system 300. The electrostatically charged misting system 300 may be any suitable configuration to produce a plurality of electrostatically charged mist particles 302. The electrostatically charged misting system 300 for producing electrostatically charged mist particles may include an outlet 304 and a charge generator 306. The outlet 304 may be a spout or nozzle or any suitable configuration to introduce a plurality of mist particles therefrom. For example, the outlet can be configured to introduce a plurality of mist particles into a growth environment, as described elsewhere herein. In some embodiments, the outlet 304 may be an ultrasonic misting spray nozzle, an ultrasonic misting puck, a high-pressure misting nozzle, or a high velocity air sprayer. The outlet 304 may atomize a liquid 308, e.g., liquid water, into a plurality of mist particles. The outlet 304 may control the size of the plurality of mist particles. For example, the outlet 304 may comprise an aperture which can be configured (e.g., adjusted) to control the size of the mist particles. The plurality of mist particles 302 (and subsequently the plurality of charged mist particles) may have a diameter ranging in size from 0.01 μm to 100 μm, alternatively, a diameter ranging from 1.0 μm to 50 μm. The mist particles 302 may be deposited on a growth matrix and growing mycelium within a growth environment, respectively stimulating either new extra-particle aerial mycelial growth or further already growing aerial mycelial growth from the growth matrix. For example, the mist can be deposited on the growth matrix surface 18 or growing extra-particle aerial mycelium surface, or, alternatively, be attracted generally to proximal location being within 5 ft of the growth matrix, 3 ft of the growth matrix, 1 ft of the growth matrix, or 6 inches of the growth matrix, based on at oppositely charged material or structure at that location. In some further embodiments, mist particles 302 may be deposited on a growth matrix and growing extra-particle aerial mycelium within a growth environment, delivering a water or a mixture or solution to the growth matrix and/or growing extra-particle aerial mycelium. In some embodiments, the plurality of electrostatically charged mist particles 302 may be introduced to a growth environment to regulate the relative humidity of the growth environment or to supplement a mist or fog that independently regulates the relative humidity of the growth environment. In some embodiments, the electrostatically charged misting system 300 may include two or more outlets 304, such that they are positioned at various locations about a large growth environment (such as a commercial scale growth environment).

The liquid 308 can be supplied to the outlet 304 from any suitable liquid source, such as a facility supply. In some embodiments, the outlet 304 may be in liquid communication with a pump system 310. The pump system 310 may supply the liquid 308 to the outlet 304 from a liquid source 312. In some further embodiments, the pump system 310 may be a syphon system. The liquid source 312 can comprise a reservoir within a container. The liquid source may supply the liquid 308 to the outlet 304 at a supply pressure (e.g., from pump system 310), for example, at the pressure of commercially viable mist-related pump devices. In some embodiments, the liquid 308 may be tap water, purified water (such as water purified by reverse osmosis or another purification method), and/or a defined medium solution. A defined medium solution may include buffer solutions. In some embodiments, the liquid 308 may be a mixture or solution that contains one or more of fertilizers, nutrients, growth factors, hormones, antioxidants, pesticides, integrated pest management sprays, fine powders, microorganisms, dyes, flavonoids, and terpenes. In some embodiments, the liquid 308 may comprise a charged component, such as an ionic component.

In some instances, the liquid may contain a cleaning, sanitizing, sterilizing, and/or decontaminating composition. The composition can be adapted to provide selective cleaning, sanitizing, sterilizing, and/or decontaminating functionality, such that neither the fungal mycelium nor the substrate on which the mycelium grows, is negatively impacted. Examples of the composition that can be adapted to provide selective cleaning, sanitizing, sterilizing, and/or decontaminating functionality include hydrogen peroxide and/or chlorine dioxide (and other chlorine moieties). In some embodiments, the composition that can provide cleaning, sanitizing, sterilizing, and/or decontaminating functionality may be administered to the mycelium and/or substrate prior to or after placement of the fungal inoculum or growth matrix in the growth environment. Other examples of composition that can be adapted to provide cleaning, sanitizing, sterilizing, and/or decontaminating functionality may include oxidant moieties that do not leave residual chemistries where growth organisms are not yet in the growth environment. In some embodiments, the composition that can be adapted to provide cleaning, sanitizing, sterilizing, and/or decontaminating functionality can be referred to collectively as “treatment agents.”

In some embodiments, the liquid 308 may be a disinfectant and/or a cleaning treatment solution to be dispensed into a growth environment. For example, a disinfectant or cleaning solution may be used to clean and/or sterilize a growth environment immediately following an incubation time period. Disinfectants may include hydrogen peroxide, chlorine dioxide, and/or similar biocidal agents.

The charge generator 306 may be any configuration suitable to impart a charge to a plurality of mist particles and thus generate a plurality of electrostatically charged mist particles. The charge generator 306 may comprise an induction charging ring disposed at the outlet 304. Alternatively, the charge generator may comprise an induction charging mesh or screen. The induction charging ring in one embodiment, may include a center pin that acts as a first electrode and a surrounding ring that acts as a second electrode. For example, the center pin may be an anode, and the ring may be a cathode. The charge generator 306 may be brought to a charge voltage. The induction charging ring may be brought to the charge voltage such that there is an electrical potential between the center pin and the ring equal to the charge voltage, generating an electromagnetic field between the center pin and the ring. In a first embodiment, the charge voltage may be between 0.01 kV and 50.0 kV. In some embodiments, the charge voltage may be between −50.0 kV and 50.0 kV.

In some embodiments, a charge generator may generate a charge. For example, a charge generator may be a charged surface or charged plate, the charge being either an opposite charge from the charge of the electrostatically charged mist or the same charge. In some embodiments, a charge generator may generate a charge, the charge imparted onto a plate or surface that is in electrical communication with the charge generator. Such charges may be implemented either continuously or by being pulsed on or off, to move electrostatically charged mist particles in a desired direction in a growth environment. Without being limited to any particular theory, the alternative charge generator may generate an electrically charged field that interacts and propels (in the case of similarly charged mist particles) or pulls electrostatically charged mist particles (in the case of oppositely charged mist particles) in a desired direction. For example, a charge generator can be positioned adjacent to a source of the electrostatically charged mist, thereby propelling the electrostatically charged mist away from the electrostatically charged mist source and towards a desired location, such as a growing extra-particle aerial mycelium or a growth matrix. In some embodiments, more than one charge generator can be placed within a growth environment such as to propel similarly charged electrostatic mist, or pull oppositely charged electrostatic mist a desired distance. For example, a plurality of charge generators can be placed in a series of regularly spaced distance intervals, wherein each of the plurality of the charge generators can be turned on in series to emit a similar charge as mist, or an opposite charge from the electrostatically charged mist such as to attract or propel the electrostatically charged mist in a desired direction. The same effect may be accomplished by pulsing a charge generator on and off with a particular voltage generating the desired charge. For example, pulsing a charge generator may create a linear motion to move airborne masses of mist in a linear direction. By way of further example, a series of electrical plates or surfaces can be placed in serial and be in electrical communication with the charge generator such that the charge generator can pulse on and off each of the series of electrical plates or surfaces individually, or in groups, along the series, thereby propelling a mass of airborne mist across the length of the series of electrical plates or surfaces.

In some embodiments, the charge generator 306 may be a corona charging unit or a conductive charging unit. For example, the outlet 304 may be brought to a charge voltage to conductively charge the liquid 308 as it exits the outlet 304. In such embodiments, the charge generator 306 and the outlet 304 may be the same component. In some embodiments, two or more charge generators 306 may be employed, and the two or more charge generators may be an induction charging ring, a conductive charging unit, and/or a corona charging unit.

The charge generator 306 may be disposed at the distal end of the outlet 304 and positioned such that the plurality of mist particles passes through the charge generator 306 after being dispensed from the outlet 304. The charge generator 306 may be brought to the charge voltage such that an electrostatic charge is imparted on the plurality of mist particles by the electromagnetic field, creating the plurality of electrostatically charged mist particles 302. In turn, the plurality of electrostatically charged mist particles 302 may be attracted to neutral or oppositely charged surfaces. In some embodiments, the electromagnetic field may impart the electrostatic charge on the plurality of mist particles 302 by depositing electrons on the surface of the mist particles. The electrostatic charge may be proportional to the charge voltage. In some embodiments, the plurality of charged mist particles 302 may have a charge to mass ratio within the range of 0.05 to 2.0 C/kg.

The plurality of electrostatically charged mist particles 302 may be directed onto a growth matrix 314. The growth matrix 314 may be positioned in a container 316, on a mycological web, or other support structure, as discussed above. Because the plurality of electrostatically charged mist particles have an electrostatic charge, they may repel each other and more evenly coat any exposed surfaces of the growth matrix 314 or extra-particle aerial mycelium growing thereupon, which may be an electrically neutral (i.e., uncharged) surface. As discussed in greater detail below, in some embodiments, the growth matrix 314 (or alternatively the support structure, adjacent material or structure, or combination thereof) may also be charged to attract or repel the plurality of electrostatically charged mist particles 302 depending on the charge of the growth matrix 314, support structure, or adjacent material (such as for example a metal plate or foil). In some embodiments, the deposition of the liquid 308 via the plurality of charged mist particles 302 may alter the characteristics of the growth matrix 314. For example, the liquid 308 may increase a growth rate of the mycelium in or on the growth matrix, or the height of the growing extra-particle aerial mycelium in certain desired locations if the liquid is targeted to target areas. If the liquid is repelled in certain locations because of similar charges associated with the growth matrix, certain growth levels may remain relatively static.

The electrostatically charged misting system 300 may direct the plurality of electrostatically charged mist particles 302 onto the growth matrix 314 throughout (e.g., continuously throughout) an incubation time period. In some embodiments, the electrostatically charged misting system 300 may periodically generate the plurality of electrostatically charged mist particles 302 during the incubation time period. For example, for a first time portion of the incubation time period, the electrostatically charged misting system 300 may generate the plurality of electrostatically charged mist particles 302, and during a second portion of the incubation time period, the electrostatically charged misting system 300 may not generate any mist, or vice versa. In some embodiments, the electrostatically charged misting system 300 may periodically generate the plurality of electrostatically charged mist particles 302 during desired portions of the incubation time period, or during desired portions of the mycelial growth. In some embodiments, the electrostatically charged misting system 300 may be configured to direct the plurality of electrostatically charged mist particles onto the growth matrix 314 based on one or more environmental conditions present in a growth environment. For example, the electrostatically charged misting system 300 may generate the plurality of electrostatically charged mist particles 302 while the relative humidity in a growth environment is above or below a humidity threshold, such as 90%, 95%, 98%, 99%, etc. or alternatively, when a temperature is above or below a certain threshold.

In some embodiments, the electrostatically charged misting system 300 may be configured to periodically alter the charge of the plurality of electrostatically charged misting particles 302. For example, the amount of electrical potential of the charge may be altered, and/or the charge may be altered during different time periods, relative to each other. For example, the electrostatically charged misting system 300 may direct the plurality of electrostatically charged mist particles 302 onto the growth matrix 314 with a first charge during a first time portion of an incubation time period, and then direct the plurality of electrostatically charged mist particles 302 onto the growth matrix 314 with a second charge during a second time portion of an incubation time period. The charge of the first and second charges may be positive, negative or neutral. In some embodiments, the charge of the first charge and the second charge can be the same or different. The charge of the first and second charges may be positive, negative, or neutral. In some embodiments, the charge of the second charge may be the same as, or different from (e.g., the opposite of) the charge of the first charge. The first and the second charge can be the same voltage, or a different voltage, relative to each other. The electrostatically charged misting system 300 may periodically introduce the plurality of electrostatically charged mist particles 302 and alter the charge of the plurality of electrostatically charged mist particles according to at least some embodiments.

In some embodiments, the electrostatically charged misting system 300 may increase or decrease the production of the electrostatically charged mist particles 302 throughout a time period within the mycelial growth, or a portion thereof, such as for instance, the incubation time period. For example, the production rate of the electrostatically charged mist particles 302 may be gradually increased as the incubation time period elapses. In some embodiments, the charge may be decreased, alternated, modulated, etc. throughout the incubation time period or a portion thereof.

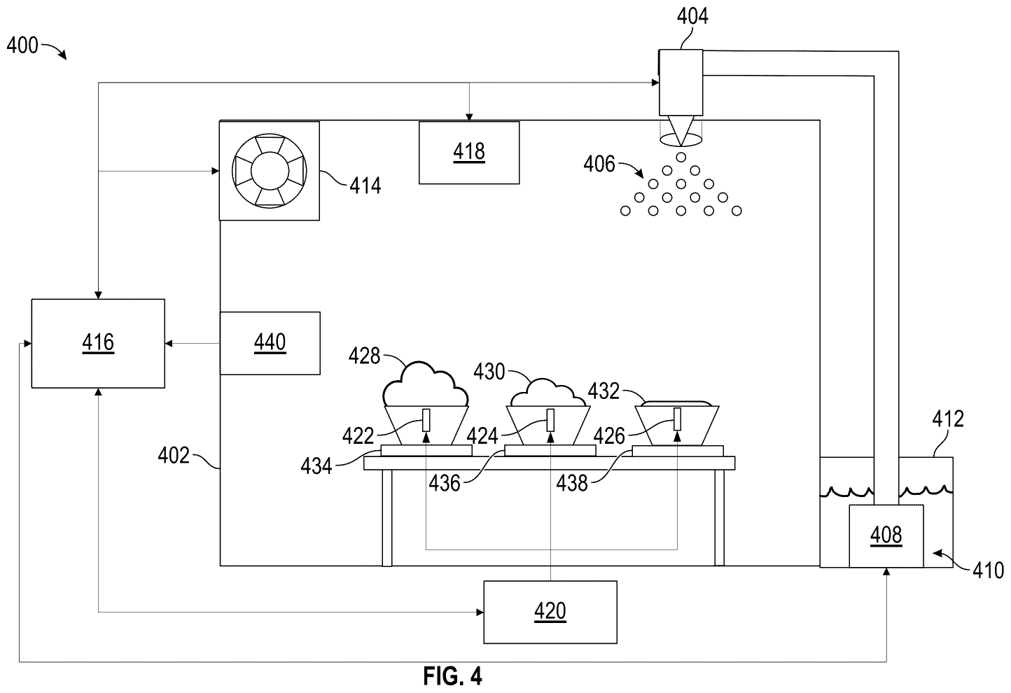

FIG. 4 illustrates an exemplary system 400 for growing or producing extra-particle aerial mycelium. The system 400 may include a growth environment 402. As defined above, the growth environment 402 may be an environment that supports the growth of mycelia, and eventually extra-particle aerial mycelium (and ultimately aerial mycelium). For example, the growth environment 402 may be a growth chamber, a growth room, an incubation chamber, or a bench top bioreactor (BTBR).

The system 400 may also include an electrostatically charged misting system 404 that may produce a plurality of electrostatically charged mist particles 406. The electrostatically charged misting system 404 may be connected to a liquid source 412, such as pump system 408. Similar to the electrostatically charged misting system 300, the electrostatically charged misting system 404 and the pump system 408 may be configured to atomize a liquid 410 and generate the plurality of electrostatically charged mist particles 406. For example, the electrostatically charged misting system 404 may include an outlet and an induction charging ring, or any other suitable configuration to generate a plurality of electrostatically charged mist particles as described above. The outlet may atomize the liquid 410 into a plurality of mist particles, and the plurality of mist particles may pass through the induction charging ring which may impart a charge to the plurality of mist particles, creating the plurality of electrostatically charged mist particles 406.

As discussed above, the liquid 410 may be water, purified water, and/or a defined medium solution. The liquid 410 may include fertilizers, nutrients, growth factors, hormones, antioxidants, pesticides, integrated pest management sprays, fine powders, microorganisms, dyes, flavonoids, terpenes and/or other treatments. The liquid 410 may be disposed in a liquid source 412, pumped from the liquid source 412 to the charged misting system 404, and atomized and charged to create the plurality of charged mist particles 406. In some embodiments, the system 400 may include two or more liquids, which may be supplied from (e.g., stored in) corresponding liquid sources. The two or more liquids may be mixed together to form the plurality of electrostatically charged mist particles 406 or may be distributed separately as two separately charged particle mists. In some embodiments, the system 400 may generate a first plurality of electrostatically charged mist particles from a first liquid and generate a second plurality of electrostatically charged mist particles from a second liquid. The first plurality of electrostatically charged mist particles and the second plurality of electrostatically charged mist particles can be introduced into the growth environment simultaneously or at different times, relative to each other. The first plurality of electrostatically charged mist particles and the second plurality of electrostatically charged mist particles can be introduced into the growth environment 402 through the same outlet, or through a first and second corresponding outlet, respectively, relative to each other.

The system 400 may also include one or more airflow units 414. The one or more airflow units 414 may be an HVAC unit, ceiling fan(s), wall-mounted fan(s), oscillating fan(s), vertical circulation fan(s), blower fan(s), squirrel cage fan(s), and/or barrel fan(s). The one or more airflow units 414 may provide an airflow at an airflow velocity or an airflow volume, which are described above. The one or more airflow units 414 may be disposed within the growth environment 402, and may be provided with independent airflow direction and/or velocity control. The one or more airflow units 414 may mix and homogenize the air within the growth environment 402. In some embodiments, the one or more airflow units 414 may be positioned within the growth environment 402 to provide homogenous airflow within the growth environment 402. In one embodiment the airflow from one or more airflow units in the growth environment may be coordinated with the actions of the electrostatically charged misting system or apparatus.

The system may further include one or more processors 416. The one or more processors 416 may be placed in electronic communication with the electrostatically charged misting system 404, the pump system 408, and/or the one or more airflow units 414, and or other components of the system 400. The processors 416 can be implemented to control any of the functionality described herein with respect to any of the components and systems described herein, including FIGS. 3 and 4, and other figures, other systems, and other components. For example, the one or more processors 416 may be communicating with sensors, and ultimately control (through a controller(s)) oxygen (O₂) content, carbon dioxide content (CO₂), temperature, humidity, misting (and charge or deposition variables associated therewith), and/or other environmental conditions within the growth environment 402. According to some embodiments, the one or more processors 416 may receive control inputs via a user interface. The user interface may enable a user to dictate the environmental conditions within the growth environment 402. In some embodiments, the one or more processors 416 may control the environmental conditions within the growth environment based on data and/or signals received from the one or more sensors 440 as discussed below.

As noted, the one or more processors 416 may also control the electrostatically charged misting system 404 and the generation of the plurality of electrostatically charged particles 406. In some embodiments, the one or more processors 416 may instruct the electrostatically charged misting system 404 to periodically generate the plurality of electrostatically charged mist particles 406. For example, for a first time portion of the incubation time period, the one or more processors 416 may instruct the electrostatically charged misting system 404 to generate electrostatically charged mist, to not generate any mist whatsoever or to generate mist, but without generating electrostatically charged mist, and during a second time portion of the incubation time period, the one or more processors 416 may instruct the electrostatically charged misting system 404 to generate electrostatically charged mist, to not generate any mist whatsoever or to generate mist, but without generating electrostatically charged mist. Alternatively, the one or more processors 416 may instruct the electrostatically charged misting system 404 to change the charge of one or more mists, in accordance with a changed environmental condition, or observed level of extra-particle aerial mycelial growth.

In some embodiments, the one or more processors 416 may instruct the electrostatically charged misting system to direct the plurality of electrostatically charged mist particles based on one or more environmental conditions present in a growth environment, such as those detected by the one or more sensors 440. For example, the electrostatically charged misting system 300 may generate the plurality of electrostatically charged mist particles 302 while the relative humidity in a growth environment is above or below a humidity threshold, such as 90%, 95%, 98%, 99%, etc. or above or below a temperature threshold. For example, if the relative humidity exceeds the threshold, the one or more processors 416 may instruct the electrostatically charged misting system 404 to stop producing electrostatically charged mist particles.

In some embodiments, the one or more processors 416 may alternate or change the charge of the plurality of electrostatically charged mist particles 406. For example, the one or more processors 416 may instruct the induction charging ring of the charged mist system 404 to generate a first electrical potential during a first time portion of an incubation time period to impart a first charge to a first portion of the plurality of electrostatically charged mist particles 406. During a second time portion of the incubation time period, the one or more processors 416 may instruct the induction charging ring of the charged mist system 404 to generate a second electrical potential to impart a second charge to a second portion of the plurality of electrostatically charged mist particles 406. In some embodiments, the one or more processors 416 may control a size of the plurality of electrostatically charged mist particles 406, a charge-to-mass ratio of the plurality of electrostatically charged mist particles 406, and/or a production rate of the plurality of electrostatically charged mist particles 406. For example, the one or more processors 416 may instruct the pump system 408 to increase a pump speed to increase a volume of the liquid 410 to the electrostatically charged misting system 404, resulting in an increased production rate of the plurality of electrostatically charged mist particles 406.

In some embodiments, the one or more processors 416 may instruct the electrostatically charged misting system 404 to gradually increase or decrease the production of the electrostatically charged mist particles throughout the incubation time period or portion thereof. For example, the production rate of the electrostatically charged mist particles 406 may be increased as the incubation time period elapses, or subsequently decreased.

The one or more processors 416 may control an airflow within the growth environment 402. In some embodiments, the one or more processors 416 may control the speed or volumetric airflow rate of the one or more airflow units 414 to generate airflow within the growth environment 402 or to select locations concurrently with directing particularly charged particles and coordinated-charged substrate locations, such as to attract particularly-charged particles. In some embodiments, the one or more processors 416 may alter the airflow within the growth environment 402 based on the functionality of other components and/or data received from sensors, imaging devices 418, or the like. In some embodiments, the speed or volumetric airflow rate of one or more airflow units 414 can be adjusted to affect the growth of the extra-particle aerial mycelium and/or mycelium. For example, the one or more processors 416 may determine by select locations within the growth environment 402 where the extra-particle aerial mycelium is growing as heterogeneous bulbous structures. Then the one or more processors may respond by adjusting the airflow or charge control (to increase deposition/attraction of mist to certain locations) within the growth environment 402, in order to cause the extra-particle aerial mycelium to grow uniformly. By way of further example, the one or more processors 416 may determine by select locations within the growth environment 402, that the electrostatically charged misting system 404 is producing the plurality of electrostatically charged particles 406. For example, such determination may be made by a strategically positioned charge detector. In an alternative embodiment, a detection of the density of charges (surface charges) at particular locations, may provide an indication via a processor, of the “Realized Mist Deposition Rate” at select locations within the growth environment 402. Accordingly, following such detection and/or associated calculation of charge densities, the one or more processors 416 may turn on or increase the speed of the one or more airflow units 414 to circulate the plurality of electrostatically charged mist particles 406 within the growth environment 402, or at select locations within the growth environment 402.

In some embodiments, the one or more processors 416 may be placed in electronic communication with a feedback device, configured to send an input signal to the processor to monitor the growth process and/or the environmental conditions within the growth environment 402. In some embodiments, the processors 416 can send an output signal, for example, in response to a corresponding input signal, to control (e.g., adjust) other components within the system 400.

For example, in some embodiments, the system 400 can include an imaging device 418. The imaging device 418 may be a camera or the like. The imaging device 418 may be disposed within the growth environment 402 and be used to monitor and alert on growth, or lack thereof, or alternatively, on undesirable heterogeneity of growth of the extra-particle aerial mycelium. In some embodiments, the imaging device 418 is used to monitor extra-particle aerial mycelium growth and to provide collected data as feedback to the system 400. For example, the feedback can be immediate. For example, unsatisfactory extra-particle aerial mycelium can be detecting by data collected by the imaging device 418, the image data can then be processed by one or more processors 416 to alert the system 400 of the unsatisfactory growth, and thereafter the environmental conditions of growth environment 402 can be adjusted to reestablish satisfactory growth of the extra-particle aerial mycelium.

In some embodiments, the one or more processors 416 may be placed in electronic communication with a voltage regulator 420. The voltage regulator 420 may control a charge of a growth medium and/or growth matrix. The voltage regulator 420 may be electronically connected to one or more growth electrodes. As used herein, a “growth electrode” refers to a material that facilitates the transfer or redistribution of electrons such as to charge a mass of a solid, liquid or gas. For example, a growth electrode can charge mist by ionizing the surrounding air through emission of an electrical field or similar mechanisms. The growth electrode may be a gold-plated or other suitable electrode to impart the desired charge. In FIG. 4, for example, the voltage regulator may be connected to a first growth electrode 422, a second growth electrode 424, and a third growth electrode 426. The first growth electrode 422 may be disposed within the substrate of a first growth matrix 428 (shown with extra-particle aerial mycelium thereupon); the second growth electrode 424 may be disposed within the substrate of a second growth matrix 430 (shown with a lesser amount of extra-particle aerial mycelium thereupon relative to 428); and the third growth electrode 426 may be disposed within the substrate of a third growth matrix 432. The voltage regulator 420 may control a voltage of the first growth electrode 422, the second growth electrode 424, and/or the third growth electrode 426. The respective growth electrodes may be controlled/regulated together (uniformly) or independently. As will be appreciated by a skilled artisan in the mycelium cultivation industry, a growth electrode can be disposed within the substrate of a growth matrix, but is not limited to such placement For example, in some embodiments, a growth electrode may be placed in any suitable way to provide electronic communication with (and thus control the charge of) an extra-particle aerial mycelium of a growth matrix, a growth matrix and/or container (or support structure), a mycological growth web, directly, or through one or more other intervening component(s) so as to attract electrostatically charged mist particles (based on opposite charge attraction).

In some embodiments, the one or more processors 416 may instruct the voltage regulator 420 to control the voltage of one or more growth electrodes. In some embodiments, the one or more processors 416 may control the voltage of the one or more growth electrodes to direct deposition of the plurality of charged mist particles 406 within a growth medium or a growth matrix. For example, the one or more processors 416 may instruct the voltage regulator 420 to set the voltage of the first growth electrode 422, the second growth electrode 424, and the third growth electrode 426. As discussed above, the plurality of electrostatically charged mist particles 406 may be attracted to neutral or oppositely charged surfaces. In some embodiments, the one or more processors 416 may instruct the voltage regulator 420 to set the voltage of the first growth electrode 422, the second growth electrode 424, and the third growth electrode 426 based on the charge of the plurality of electrostatically charged mist particles. For example, the first growth electrode 422, the second growth electrode 424, and the third growth electrode 426 may be set to a positive charge if the plurality of charged mist particles 406 are negatively charged.

The one or more processors 416 may periodically alter the charge of the growth electrodes 422, 424, and/or 426. For example, the one or more processors 416 may alter the charge of the growth electrodes 422, 424, and/or 426 between a negative charge, a positive charge, and uncharged during an incubation period or portion thereof. In some embodiments, the one or more processors 416 may alter the charge of the growth electrodes 422, 424, and/or 426 based on a desired alteration in the charge of the plurality of electrostatically charged mist particles 406. For example, if the plurality of electrostatically charged mist particles 406 is altered between charged and uncharged, the one or more processors 416 may correspondingly alter the charge of the growth electrodes 422, 424, and/or 426 between an opposite charge and uncharged. Similarly, if the charge of the plurality of electrostatically charged mist particles 406 alters between positive and negative, the one or more processors 416 may correspondingly alter charge of the growth electrodes 422, 424, and/or 426 between negative and positive, if the desired result is the increased deposition or more uniform deposition of mist on the growth matrix or growing extra-particle aerial mycelium. In some embodiments, the one or more processors 416 may alter the charge of the one or more growth electrodes based on data received from the imaging device 418 or other sensor information.

Based on the images and/or data received from the imaging device 418, the one or more processors 416 (or human manager at a user interface) may instruct the voltage regulator 420 to set the first growth electrode 422 at a first charge, the second growth electrode 424 at a second charge, and the third growth electrode 426 at a third charge. Returning to the example, the one or more processors 416 may set the voltage of the third growth electrode 426 at a maximum capacity voltage based on the determination that the third growth matrix 432 has demonstrated no growth or poor growth. The one or more processors 416 may set the voltage of the second growth electrode 424 to a 50% capacity voltage and set the voltage of the first growth electrode 422 to zero. The voltage of each of the growth electrodes 422, 424, and/or 426 may be opposite to the charge of the plurality of electrostatically charged mist particles 406. These voltages may allow the plurality of electrostatically charged particles 406 to accumulate more on the third growth matrix 432 than the second growth matrix 430 and first growth matrix 428 and to accumulate more on the second growth matrix 430 than the first growth matrix 428. In turn, the first growth matrix 428, the second growth matrix 430, and the third growth matrix 432 may become substantially homogenous by the end of an incubation or desired time period. The one or more processors 416 or human manager at a user interface, via one or more controllers, may alter the charge, particle size, charge-to-mass ratio, or production rate of the plurality of electrostatically charged mist particles 406 as a response based on the images and/or data received from the imaging device.

The system 400 may also include one or more scales. The one or more scales may be placed underneath a container or support structure that houses or supports a growth media or growth matrix. The one or more scales may be configured to monitor the weight of the growth media or growth matrix throughout the incubation period. The weight of the growth medium or growth matrix (and growing mycelium) may change, such as as carbon and moisture content increases or decreases with mycelial growth and the fungal respiration occurs. For example, as shown in FIG. 4, a first scale 434 may be disposed underneath the first growth matrix 428; a second scale 436 may be disposed underneath the second growth matrix 430; and a third scale 438 may be disposed underneath the third growth matrix 432. In some embodiments, scales may be placed under two or more containers, three or more containers (or support structures), four or more containers (or support structures), etc. Scales may also be placed underneath shelves that house the containers in the growth environment, such as the shelves 240 shown in FIG. 2.

The one or more processors 416 may be in electronic communication with the one or more scales. For example, the one or more processors may be in electronic communication with the first scale 434, the second scale 436, and the third scale 438. In some embodiments, the one or more processors 416 may alter the voltage of the one or more growth electrodes based on the weight data received from the one or more scales. Similar to the example above, the one or more processors 416 may receive a first weight of the first growth matrix 428 from the first scale 434, indicating good growth. The one or more processors 416 may receive a second weight of the second growth matrix 430 from the second scale 436, indicating moderate growth, and may receive a third weight of the third growth matrix 432 from the third scale 438, indicating poor growth. Based on the first weight, second weight, and third weight, the one or more processors 416 may set the voltage of the first growth electrode 422, second growth electrode 424, and third growth electrode 426 to 0%, 50% capacity, and 100% capacity, respectively. The one or more processors 416 may alter the charge, particle size, charge-to-mass ratio, or production rate of the plurality of electrostatically charged mist particles 406 based on the weight data received from the first scale 434, the second scale 436, and/or the third scale 438.

The one or more processors 416 may also continuously update the voltage of a corresponding growth electrode based on an updated weight. For example, after the first growth matrix 428, the second growth matrix 430, and the third growth matrix 432 become substantially homogenous due to the disparate growth electrode charging described above, the one or more processors 416 may set the voltage of the first growth electrode 422, the second growth electrode 424, and the third growth electrode 426 to the same value. In some embodiments, the one or more processors 416 may set the voltage of the one or more growth electrodes based on image(s) and/or data received from the imaging device 418 and weight data received from the one or more scales.

The system 400 may also include other feedback devices, such as one or more sensors 440. For example, the one or more sensors 440 may include a CO₂ sensor, an O₂ sensor, a temperature sensor, a relative humidity sensor, a pressure sensor, an airflow sensor, or the like. The one or more sensors 440 may monitor the growth process and/or the corresponding detected environmental condition(s) within the growth environment 402.

The one or more sensors 440 may be placed in electronic communication with and/or send an input signal to the one or more processors 416. The processors 416 can send an output signal, for example, in response to a corresponding input signal, to control (e.g., adjust) other components within the system 400 to a desired environmental condition. For example, in some embodiments, the one or more processors 416 may control (e.g., alter, or maintain) the charge of the first growth electrode 422, the second growth electrode 424, and/or the third growth electrode 426 in response to the input signal received from the one or more sensors 440. For example, a CO₂ sensor may detect a CO₂ value for the growth environment 402 that is out of a recommended range described above. The one or more processors 416 may increase the voltage of the one or more growth electrodes 422, 424, and/or 426 to increase deposition of the plurality of electrostatically charged mist particles. In some embodiments, the one or more processors 416 may control the charge of the one or more growth electrodes based on images and/or image data received from the imaging device, weight data received from the one or more scales, and/or data received from the one or more sensors. The one or more processors may control the charge, particle size, charge-to-mass ratio, or production rate of the plurality of electrostatically charged mist particles 406 based on images and/or image data received from the imaging device, weight data received from the one or more scales, and/or data received from the one or more sensors. In a situation where a sensor detects no deposition of electrostatically charged particle, or a higher density of charges (presumably resulting from a higher deposition of mist at a particular location), the mist apparatus can be directed to produce more mist, shift mist to a particular location, or decrease mist output.

Advantageously, the charged misting system 300 and system 400 allow for increased mist deposition as well as controlled and uniform deposition of mist and mist constituents on a growth matrix and/or growing extra-particle aerial mycelium. In turn, the extra-particle aerial mycelium generated from the growth matrix is more homogenous regardless of its position in a growth environment, the impact of structural features on airflow in the growth environment, its proximity to an electrostatically charged misting system, and in one embodiment, airflow rate disparities within a growth environment. Moreover, the size of the growth environment may be increased, and extra-particle aerial mycelium may be produced on a larger scale because the electrostatically charged mist particles may be more uniformly distributed within the growth environment and ensure homogeneity of the resulting extra-particle aerial mycelium (as a result of charge repulsion in the individual mist particles). Furthermore, targeted deposition through the use of electrostatically charged mist particles may increase yield (by providing mist where it is needed most) and reduce waste (by reducing deposition where it is not needed or needed less). The waste may be realized in the form of less energy consumption, less mist usage, and less product spoilage/chance of fruiting which might occur were too much mist be applied to a particular growth region in the growth environment.

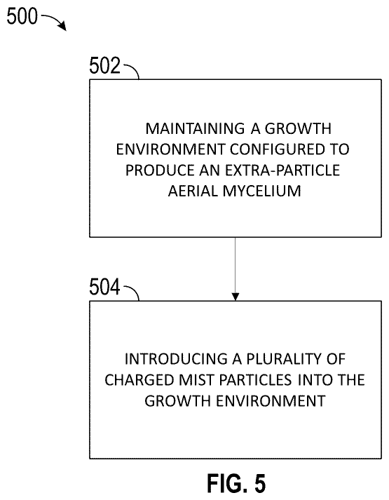

FIG. 5 illustrates an exemplary process 500 of growing an extra-particle aerial mycelium. The process 500 begins with step 502 where a growth environment is maintained to produce an extra-particle aerial mycelium. As discussed herein, a growth matrix may be placed within a growth environment. The growth environment may be configured to support the growth of mycelia.

The process 500 may move to step 504 where a plurality of electrostatically charged mist particles are introduced into the growth environment. The plurality of electrostatically charged mist particles may be introduced into the growth environment by an electrostatically charged misting system. The electrostatically charged misting system may include an outlet and a charge generator as described above. In some embodiments, the electrostatically charged misting system may draw a liquid from a liquid source via a pump system. The liquid may pass through the outlet, creating a plurality of mist particles. The charge generator may apply an electrostatic charge to the plurality of mist particles, creating the plurality of electrostatically charged mist particles. The electrostatically charged mist particles may be evenly distributed within the growth environment by nature of their charge (such as charge repulsion), and in one embodiment, also to attraction to selectively positioned growth matrix electrodes or other electrodes to attract oppositely charged mist particles so as to be drawn to, and deposited on, a growth media and/or a growth matrix, at all or select locations.

In some embodiments, following 504, the process 500 can further include monitoring one or more environmental conditions in the growth environment, the electrostatically charged mist, the growth of the extra-particle aerial mycelium, and/or changes in charge deposition. In some further embodiments, following monitoring, the process 500 can further include adjusting one or more configurations of the growth environment in response to the monitoring. For example, adjusting one or more configurations can include adjusting environmental conditions in the growth environment, adjusting the electrostatically charged mist, adjusting the growth of the extra-particle aerial mycelium, and/or adjusting charge deposition.

Furthermore, the growth environment may also include sensors to detect the electrostatically charged mist particles, which sensors can trigger actions directly, or communicate commands to processors also associated with the growth environment that could then initiate mister actions to address either: 1) absence of electrostatically charged particles at select locations within the growth environment, 2) overabundance of charge density (as a result of overabundance of mist) at select locations within the growth environment, or 3) sensed environmental conditions or weights of growth material, which could result from either too much growth or too little growth in particular locations within the growth environment.

One or more processors (or a system manager through a user interface) may be configured to control the electrostatically charged misting system and/or the growth environment. In some embodiments, the one or more processors (or a system manager through a user interface) may instruct the electrostatically charged misting system to introduce the plurality of electrostatically charged mist particles into the growth environment. The one or more processors (or a system manager through a user interface) may instruct the electrostatically charged misting system to periodically introduce the plurality of electrostatically charged mist particles into the growth environment. The one or more processors (or a system manager through a user interface) may instruct the electrostatically charged misting system to periodically change the electrostatic charge of the plurality of mist particles between a positive charge, a negative charged, and a neutral (i.e., uncharged) state. In some embodiments, a growth electrode may be embedded within a growth media and/or a growth matrix, or other location as previously noted. The growth electrode may charge the growth media and/or the growth matrix or other location as previously noted. For example, the growth electrode may positively charge a growth matrix such that a plurality of negatively charged mist particles are attracted to the surface of the growth matrix. In some embodiments, the one or more processors may control the charge of the growth electrode via a voltage regulator as discussed above.

The growth environment may include one or more imaging devices, one or more scales, and/or one or more sensors to monitor conditions of the growth environment and/or the growth of a mycelium therein, including the presence or absence of electrostatically charged mist particles on the growth matrix (or on a specific sensor), or the density of a charge on the growth matrix (or on a specific sensor). The imaging device(s), the scale(s), and/or the sensor(s) may be configured to be placed in electronic communication with the one or more processors (and/or human system manager). Based on the input from the one or more imaging devices, the one or more scales, and/or the one or more sensors, the one or more processors may instruct the electrostatically charged misting system to introduce the plurality of electrostatically charged mist particles into the growth environment. In some embodiments, the one or more processors may instruct the voltage regulator to alter the charge of one or more growth electrodes based on an input from the imaging device(s), the scale(s), and/or the sensor(s). Alternatively, the overall system may include sensors, such as humidity level sensors, thermostats, scales, or charge density-detection devices, which trigger a change in operation of the electrostatically charged mist system, such that the electrostatically charged mist is altered, by being discontinued, increased, or decreased in flow or occurrence.

### EXPERIMENTS

The following are experimental setups for demonstrating the advantages of the systems and methods disclosed herein. The experiments are not intended to limit the present disclosure but are intended to illustrate the advantages of the electrostatically charged misting systems described herein.

### Experiment 1

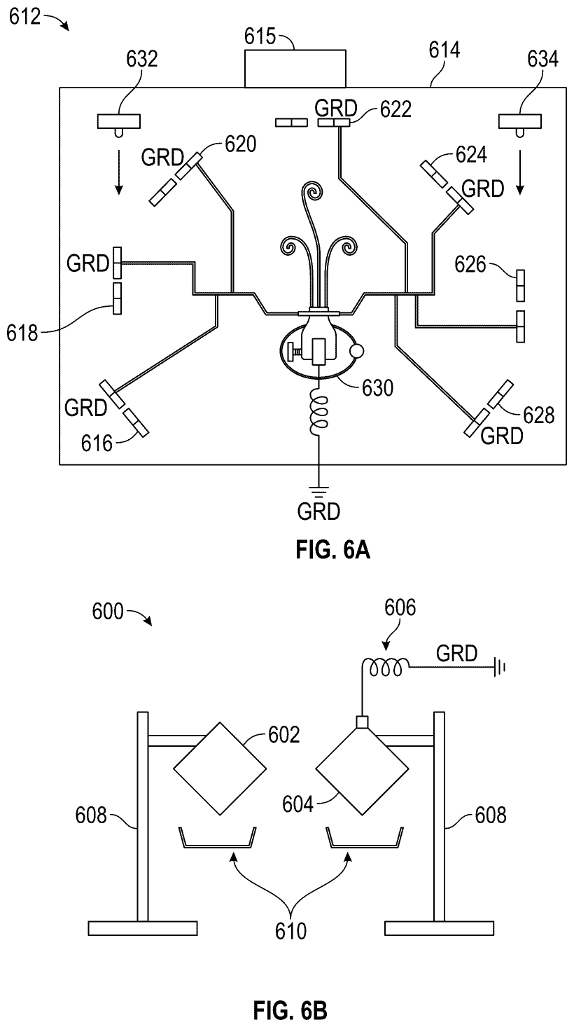

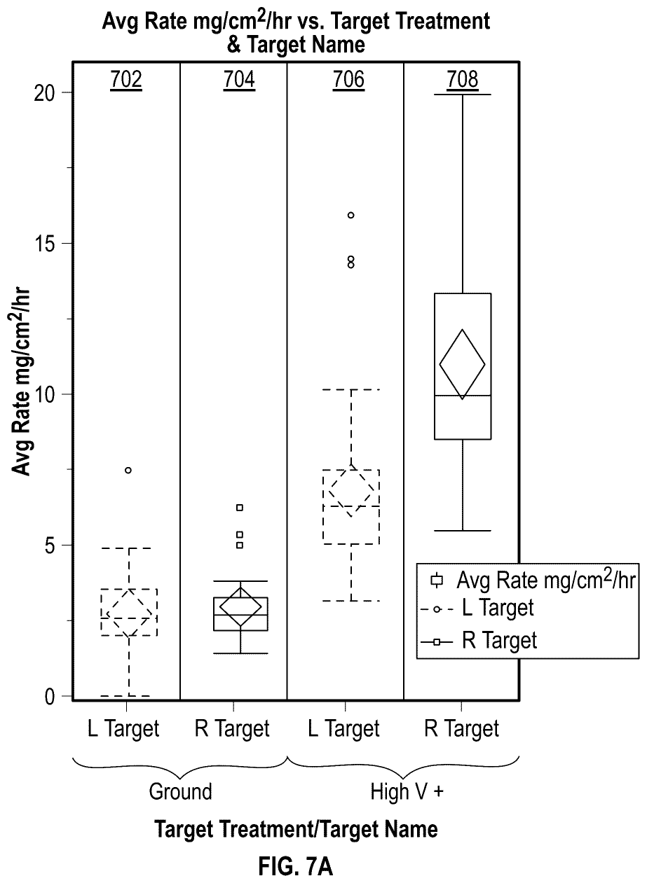

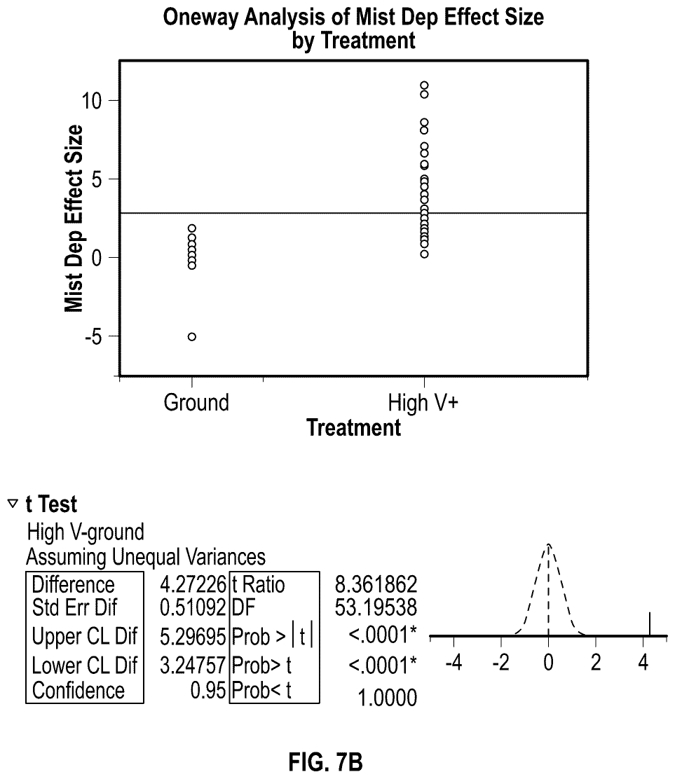

FIGS. 6A and 6B illustrate an experimental setup used to determine a deposition rate of an electrostatically charged misting system on control deposition targets and electrostatically charged deposition targets. FIGS. 7A, 7B, 8A, 8B, 9A, and 9B depict data acquired from the experimental setup shown in FIGS. 6A and 6B.

FIG. 6A illustrates an experimental setup 612 of an electrostatically charged misting system. The experimental setup 612 includes a growth environment 614 configured to regulate at least relative humidity and temperature within the growth environment. The experimental setup 612 can include seven deposition targets, which are correspondingly labeled 616, 618, 620, 622, 624, 626, and 628. Each of the deposition targets include the components shown in FIG. 6B. The deposition targets 616, 618, 620, 622, 624, 626, and 628 can be arranged in a semicircle facing a first side of the growth environment 614. The experimental setup 612 can include a charged misting system 630, such as the charged misting system in FIGS. 3 & 4. In some embodiments, the charged misting system 630 generates a negatively charged mist. The charged misting system 630 can be disposed at the center of the growth environment 614 and the semicircle of deposition targets 616, 618, 620, 622, 624, 626, and 628. The charged misting system 630 can be pointed toward a second side of the growth environment 614 opposite the first side. Additionally, the experimental setup 612 can include a first airflow unit 632 and a second airflow unit 634. The airflow units 632 and 634 can be high-velocity fans. The first airflow unit 632 and the second airflow unit 634 can be positioned along the second side of the growth environment 614 such that they blew towards the first side of the growth environment 614. In some embodiments, the experimental set up can comprise a mist generator 615. In some embodiments, a mist generator 615 can include a steam generator. In some embodiments, both a mist generator 615 and a steam generator can comprise the growth environment. For example, a mist generator 615 can be used to provide liquid water to a growing extra-particle aerial mycelium, and a steam generator can be used to adjust the relative humidity within the growth environment. In some further embodiments, “GRD” can refer to grounding. For example, one or more deposition targets can comprise or be grounded as would be understood by an electrical engineer or a skilled artisan in related fields.

Prior to a deposition test, the deposition targets 616, 618, 620, 622, 624, 626, and 628 and their associated drip collection plates can be weighed. The growth environment 614 can be brought to a target relative humidity between 0% and 100% relative humidity, such as 10%, 35%, 45%, 50%, 90%, 95%, 99%, or ≥99% relative humidity. Once the growth environment 614 reaches the target humidity, the deposition targets 616, 618, 620, 622, 624, 626, and 628 can be placed within the growth environment 614, and the electrostatically charged misting system run for 30 minutes. A charge can be applied to the charged deposition targets, such as a ground charge or positive charge (“High V+”) that is opposite from the negatively charged mist produced by the electrostatically charged misting system 630. After the 30 minutes elapsed, the electrostatically charged misting system 630 can be turned off. The deposition targets 616, 618, 620, 622, 624, 626, and 628 can be analyzed for mist deposition by (1) visual inspection, (2) calculating a difference in weight for each of the deposition targets before and after misting, and (3) calculating a difference in weight for each of the corresponding drip collection plates of the deposition targets before and after misting. In some embodiments, the deposition targets can be placed adjacent to or coupled with the extra-particle aerial mycelium, or part thereof, and/or the support structure enclosing or supporting the substrate, growth medium, growth matrix, and/or extra-particle aerial mycelium. In some further embodiments, the deposition targets are placed adjacent to the substrate, growth medium, growth matrix, and/or extra-particle aerial mycelium at a distance therefrom. For example, the distance can comprise 0.5 m, 1.0 m, 1.5 m, 2.0 m, 2.5 m, 3 m, or any value therebetween.

FIG. 6B illustrates a deposition target set 600 in greater resolution, and as shown in FIG. 6A with reference to 616, 618, 620, 622, 624, 626, and 628. The deposition target can include a first target 602 and a second target 604. In some embodiments, the targets can be electrodes. The deposition targets 602 and 604 can comprise 90 mm×90 mm aluminum foil square sheets hanging from a plastic tote. They were electrically isolated from the rest of the chamber unless connected to a ground. The first target 602 can be uncharged and represent a “control target. that is unconnected. The second target 604 can be charged via the clamp 606 and represent a “charged target.” For example, the second target 604 can be connected to a ground or a mist-opposite charge, such as a positive voltage (“High V+”) charge, where the mist can be received having a negative charge. The electrodes 602 and 604 can be suspended with a plurality of corresponding stainless steel holders 608, where contact of the stainless steel holders 608 with any portion of the growth environment can be presented with plastic insulating material coupled with, e.g., a base of the stainless steel holders. Additionally, the set of targets 600 can include a plurality of corresponding drip collection plates 610 placed underneath the targets 602 and 604. The drip collection plates 610 can be configured to capture moisture that collects on the electrodes 602 and 604 and subsequently dripped off.

As mentioned above, FIGS. 7A, 7B, 8A, 8B, 9A, and 9B depict data acquired from the experimental setup shown in FIGS. 6A and 6B. Electrostatic mist treatment were implemented to test the effect of electrostatic charging of mist on the deposition uniformity, volume and rate of airborne mist. Mist treatments consisted of electrostatically charging an electrode in the presence of electrostatically charged airborne mist. The treatments consisted of exposing the electrostatically charged airborne mist to an electrode that was connected to either a source of high voltage and/or that was grounded (as an “R” target), and also to an unconnected control target (“L” Target), as used herein, electrode and target are used interchangeably and are synonymous.

FIG. 7A is a box-and-whisker plot demonstrating mist deposition rates between different electrostatic mist treatments (in which an electrostatic charging ring, for example, is turned on, generating electrostatically charged mist). Data shown in subplots 702 and 706 were for targets that were not-connected to any electrical power source. Data shown in subplots 704 and 708 were connected to an electrical power source. Treatments labelled as “Ground” are shown in plots 702 and 704, and included use of grounded targets. Treatments labelled “High V+” are shown by the plots 706 and 708 and included use of targets that were connected to a source of high voltage.

Data demonstrate that the “Ground” treatments do not affect mist deposition rate, while the “High V+” treatments increase mist deposition for both the connected and non-connected targets. Additionally, data for 708 “High V+” treatment show significantly more mist deposition than in 706 that is not connected to an electrical power source. Results indicate that connecting targets to a “High V+” (i.e., high voltage) electrical power source increases the mist deposition rate on the charged target and to the general location of the target. Essentially, the data demonstrates a “proximate” increased electrostatically charged mist effect from the high voltage, on material/structure (L Target) within a location proximate to the high voltage charged target (R Target).

FIG. 7B is a chart demonstrating a t-test statistical analysis between the average mist deposition measurements of the “Ground” and “High V+” charged targets. Results of the t-test demonstrate that the “High V+” treatment is statistically significantly different from the control data (p-val.>0.05). Statistical analysis corroborates that the “High V+” electrical treatment has a significant effect in increasing mist deposition when compared to the “Ground” targets.

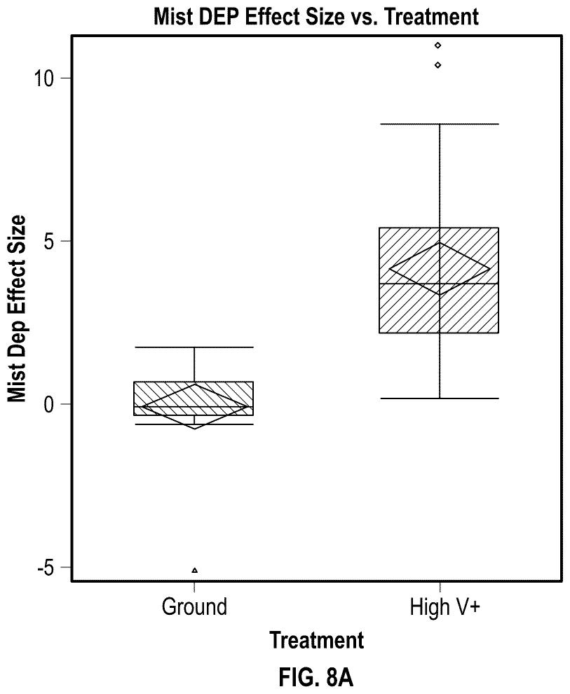

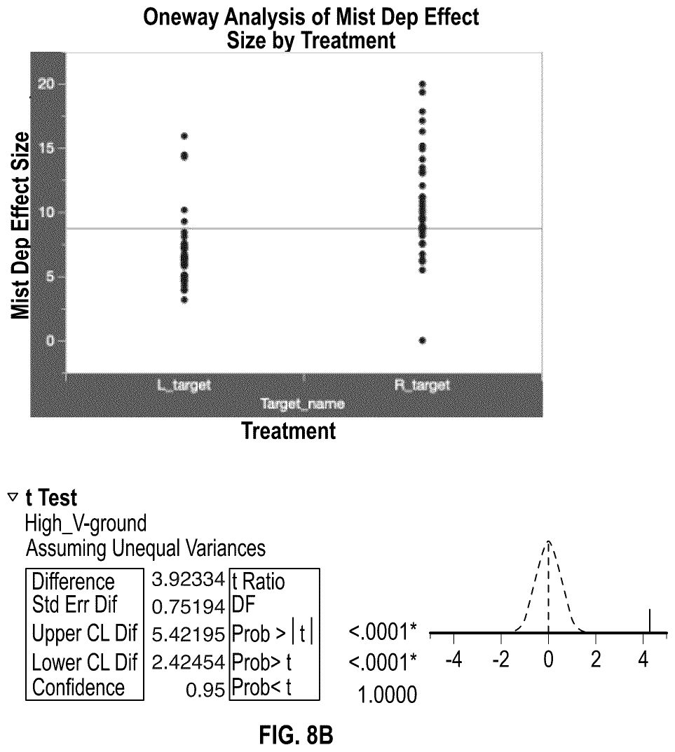

FIG. 8A is a chart demonstrating the difference in mist deposition rates between the “Ground” electrical treatment and the “High V+” electrical treatment. This graph is centered around zero and extends both in the positive and negative directions. The mean of the “Ground” treatment and the range of the box-and-whisker plot is approximately zero (0), showing that there is very little difference in deposition between the “High V+” targets and “Ground” targets when the latter are grounded.

FIG. 8B is a chart demonstrating a t-test analysis of the difference in mist deposition rates by electrical treatment V+ shown in FIG. 7A. Results of the t-test shows that there is statistical evidence supporting a difference between the “L” treatment that is not connected to an electrical power source and “R” treatment that is connected to an electrical power source. These data corroborate that “High V+” electrical treatments can increase the mean effect size of mist deposition compared to the control deposition targets.

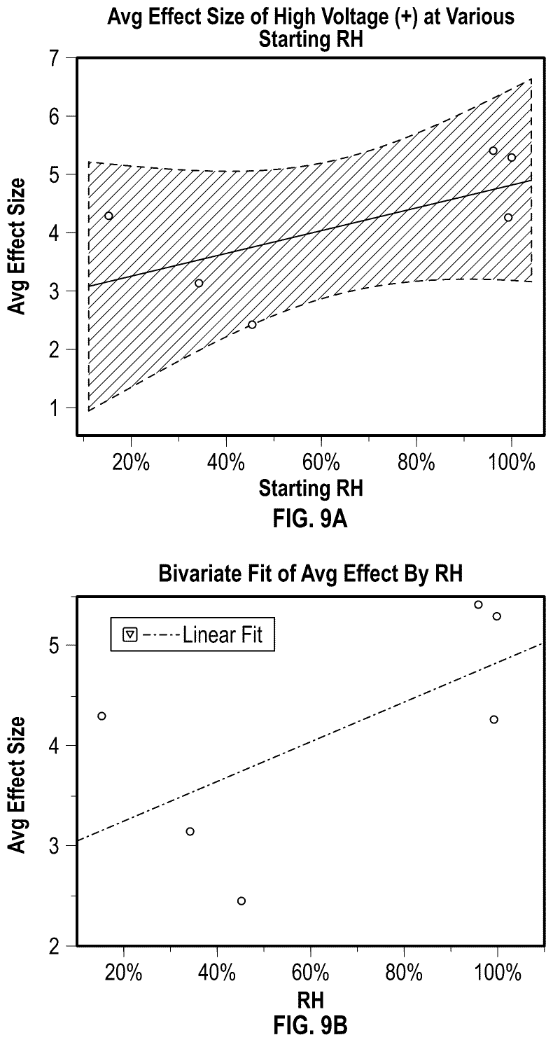

FIG. 9A is a chart demonstrating average effect size of deposition rates for the “High V+” treatment plotted by the starting relative humidity (RH). Contrary to the expected result that as starting RH increases, the average effect size of the absolute difference between no charge and a tested charge. The fully saturated air of a ≥99% RH environment was hypothesized to act as a conductor that would theoretically dissipate or reduce the charge conveyed to the electrostatic mist particles. This effect was not observed. This graph seems to show the opposite effect, where the effect size increases as the starting RH value increases, i.e. deposition of electrostatic mist on the charged target increases. Thus, increased and targeted deposition of electrostatic mist should be maintained even at high relative humidity (>95%).

FIG. 9B is a chart illustrating a linear fit statistical analysis of the effect of starting RH on the rate of mist deposition when the “High V+” electrical treatment is applied to airborne mist.

There are multiple effects of electrostatic mist deposition that can be tuned for specific goals when targeting locations within heterogenous growth environment conditions. A few results have been elucidated from the experiment described above:
1. Connecting a deposition target to a mist-opposite charge, e.g. a positive charge, where the mist is negatively charged, increases rates of mist deposition on a deposition target.
2. Connecting a deposition target to an electrical ground increases the homogeneity of mist deposition across all targets connected to that same electrical ground.
3. Increasing mist deposition rates of the deposition targets connected to the positive high voltage current are not affected by a growth chamber at 100% RH.
4. High voltage at a target also increases mist deposition in a proximate target that is not connected to the high voltage.

These results indicate the systems and methods described herein can be used to tune mist deposition with both fine and coarse levers for tunability. Coarse-tuning can correspond to positive high voltage current applied to the deposition target, thereby providing increased average mist deposition rates and an increased coefficient of variance (CV). Fine-tuning can correspond to the electrical ground connection, which provides a slight reduction in CV without any changes to average mist deposition rates.

The system and methods described herein can work to standardize growth rates and homogeneity of extra-particle aerial mycelium production by delivering more or less mist to places with deficiencies, or overabundance, and/or leveling out the deposition of mist in places that are not deficient. Sensors may be used in connection with such electrostatically charged mist systems to communicate the presence or absence of mist, or the relative abundance of such mist. Such sensors may be focused on indirect measurements, such as weights of deposited mist on detectors, or measurements to detect charges/charge density/surface charge density.

### EXAMPLES

The following are a series of example systems and methods for producing an extra-particle aerial mycelium. The examples are not intended to limit the embodiments described herein but are intended to illustrate how charged misting systems may be employed in the production of extra-particle aerial mycelium.

### Example 1

According to some embodiments, an example protocol for extra-particle aerial mycelium production using electrostatically charged mist could include the following steps:

A precolonized substrate comprising, for example, 50% soyhulls, 50% oak pellets, inoculated with 19% millet spawn of Pleurotus ostreatus may be loaded onto racks inside a growth environment.
1. The growth environment may be sealed, and the mycelium run may begin.
Environmental set points can be set to:

| i. | Temp: | 76° | F. |
|---|---|---|---|
| ii. | Relative Humidity: | 100% | |
| iii. | Mist Duty Cycle: | 15% | |
| iv. | Air Velocity: | 30 | ft/min |

2. The voltage of the electrostatic charging unit may be set such that the charge to mass ratio (CMR) of the mist water is within the range of 0.05 to 2.0 C/kg. This may be achieved by using a voltage of ˜5 kV. Four electrified commercially available misters (i.e., sometimes commonly referred to foggers, such as those sold by SuperHandy [SUPERHANDY® ELECTROSTATIC ULV FOGGER], Pelsis [DYNA-FOG HURRICANE ES®], Victory Innovations [VICTORY®], or Ikeuchi USA, Inc. [AKIMISTR], or emist [EMIST® EPIX360®], wherein the apparatus produce fine liquid droplets may be placed at four separate locations within the growth environment at about ¾ of the full height of the growth environment.
3. The substrate and subsequent growth matrices may be cultured for an incubation period, such as 13 days.
4. After the incubation period has elapsed, the power supply may be disconnected from the electrostatic fogger to reduce the risk of a shock to an operator.
5. The growth environment may be opened and the extra-particle aerial mycelium may be harvested.

### Example 2

This permutation is the same as example 1, except that instead of a positive charge being conveyed to the electrostatic charging unit, a negative charge is employed.

### Example 3

This permutation is the same as the previous examples, except that the voltage applied to the electrostatic charging unit can span from 0.01 kV to 50 kV positive or negative charge).

### Example 4

This permutation is the same as example 1, except that the electrified commercially available sprayers can be placed in various other locations. These include on the ceiling, on the walls, on the racks, next to the doors, or in-line with the HVAC air handling unit.

### Example 5

This permutation is the same as all the previous examples, except that the electrified commercially available sprayers can be substituted for any electrostatic sprayers that produce mist within a particle size range of 0.01 μm to 100 μm in diameter and convey a charge to the particles in the range of (±) 0.05 to 2.0 C/kg CMR.

### Example 6

This permutation is the same as all the previous examples, except the mist water can be substituted with any defined media solution for nutritional addition to the growth surface. Any water source can be used for this method and can be used in combination with any and all available fertilizer products approved for mushroom cultivation or other novel fertilizer recipes.

### Example 7

This permutation is the same as all the previous examples, except the mist water can be substituted with any defined media solution for growth factor/hormonal applications to the growth surface. Reverse osmosis water or any other ultra-pure water source could be used in combination with any and all commercially available growth factors/hormone products approved for mushroom cultivation.

### Example 8

This permutation is the same as all the previous examples, except the mist water can be substituted with any defined media solution for pesticide or integrated pest management (IPM) applications to the growth surface, including but not limited to products such as essential oils, nematode controls, chemical fly killers, neem oil, soap, pyrethrin, etc. Any water source can be used in combination with all commercially available pesticide or IPM products approved for mushroom cultivation.

### Example 9

This permutation is the same as all the previous examples, except that the air handler unit can be substituted or supplemented by any combination of fans. This could include, but is not limited to, oscillating fans, vertical circulation fans, ceiling fans, fixed wall mounted fans, blower fans, squirrel cage fans, barrel fans, etc.

### Example 10

This permutation is the same as all the previous examples, except that the charging method can be substituted from an induction-based charging ring to any number of electrostatic charging methods effective to achieve a charged particle within the CMR range of (±) 0.05 to 2.0 C/kg. These methods include, but are not limited to, corona charging, high velocity air speed static charging, conduction charging, etc.

### Example 11

This permutation is the same as all the previous examples, but in this scenario the electrostatic mist utilizes oxidating agents to produce oxidative stress to induce a stress response. The purpose of an induced stress response would be to trigger defense pathways present within the fungi's genome, either naturally existing or genetically engineered, to achieve a specific outcome such as pigmentation production, antibiotic production, or luciferase pathogen indicator pathways, among others.

### Example 12

This permutation is the same as all the previous examples, but in this scenario the electrostatic charging unit can be utilized to deliver a fine powder to the growth surface either instead of mist water, incorporated into the mist water, or in some combination thereof (e.g., powder, then water, in a dual duty cycle). These fine powders can be of any variety and depending on their properties could accomplish a variety of tasks, including but not limited to morphological alteration, cleaning, dehumidifying, biological delivery, etc.

### Example 13

This permutation is the same as all the previous examples, but in this scenario the electrostatic mist can be supplemented with microorganisms. These microorganisms might be deployed for the purpose of contaminate suppression, contaminate prevention, induce morphological changes, flavor modification, encourage symbiosis increasing yields, etc.

### Example 14

The electrostatic mist could be utilized to stimulate growth of the hyphal tips due to the mist's similarity to elemental ions. The positive charge of the electrostatic mist may simulate the charges of elemental ions, inducing a nutrient scavenging expression from the mycelium, causing it to change its morphological characteristics. Brand & Gow (2009) Curr. Opin. Microbiol. 12 (4): 350-357 suggest that hyphal tips are attracted to the positively charged particles and that the hyphae will grow towards them.

### Example 15

This permutation is the same as all the previous examples, but in this scenario the electrostatic mist or permutations thereof can be cycled with uncharged water or any other permutation to create a time-bound layering effect. This effect would change the material or morphological properties of the extra-particle aerial mycelium being grown in thin sections, creating layers of altered material properties which would influence the properties of the final product.

### Example 16

This permutation is the same as all the previous examples, but in this scenario the electrostatic mister would alternate between positive and negative charging currents. For example, the first application of mist water would convey a positive charge to the growth surface. Then, the electrostatic mister would deliver mist particles conveying a negative charge to the growth surface. This example is also possible in the opposite scenario, in which negative charge is applied first and the positive charge is applied second. This treatment may be achieved via a variety of methods, including utilizing the same electrostatic mister, utilizing alternating misters with opposite charges, etc. It is proposed that this application might increase mist deposition, alter morphologies, force hyphal entanglement, and otherwise contribute to the overall health and growth of the extra-particle aerial mycelium.

### Example 17

This permutation is the same as all the previous examples, but in this scenario the electrostatic mister would be utilized to also deliver supplemental ingredients during growth, including but not limited to anionic and cationic dyes, flavonoids, terpenes, etc. These compounds would alter the final properties of the extra-particle aerial mycelium product by changing the color, flavor, smell, etc.

### Example 18

Three trays of extra-particle aerial mycelium may be grown on top of a growth bed inside of a temperature- and humidity-controlled growth environment with heterogenous airflow. The electrostatic mister may be programmed with its water pump on a duty cycle to spray charged mist water from a pure water liquid source. The trays and the growth matrices therein may all have variable amounts of growth due to heterogeneity of mist deposition within the large chamber. Due to the variance in growth observed via a camera, changes to the voltages of the multi-channel high voltage positive (±) regulator depending on the growth levels may be made, either by a systems administrator or processor. The voltage may be conveyed to the growth substrate and extra-particle aerial mycelium via gold plated electrodes buried in the substrate. The tray with the most growth may receive little to no voltage current. The tray with average growth may receive medium capacity voltage relative to the other two. The tray with poor growth may receive the maximum amount of voltage because it has the poorest growth of all the trays in the whole chamber and needs more mist to be delivered in order to catch up to the other two trays. The operator or processor may update its voltages each hour depending on the respective growth heights of the trays.

### Example 19

This example is nearly the same as scenario 18, except for the camera which is used to provide information to an operator or processor. The camera may be replaced with scales placed on top of the growth bed, but underneath each growth tray. A CO₂ sensor may also be placed inside of the growth environment. The current weights of the combined growth tray (or support structure), substrate, and extra-particle aerial mycelium may be referenced against a starting weight and the CO₂ concentration within the growth environment. Baselines of each may be established at the beginning of the growth cycle. Weight of this combination can be expected to change over time in predictable ways. Carbon mass can be expected to decrease in the substrate via the release of CO₂ due to the fungal metabolism converting carbon into sugars and CO₂. Water mass of the combination can be expected to increase over time as more mist is deposited onto the substrate and extra-particle aerial mycelium. Estimates of growth rates for each tray of extra-particle aerial mycelium may be developed, which then informs operator decisions on actuation of variable voltages in the multi-channel high voltage positive (+) regulator. The operator or processor may supply the highest voltage to the slowest growing tray, the weakest voltage to the fastest growing tray, and may provide proportional voltages to all the trays with growth rates that fall in between these two. The largest amount of electrostatically charged mist may be deposited onto/or otherwise supplied to the growth tray (or support structure) with the highest voltage and the rest of the growth trays (or support structures) may receive amounts of electrostatically charged mist proportional to the voltages supplied to their substrates.

### Example 20

This example is the same as scenarios 18 and 19, except that all three sensors (camera, high sensitivity weight scales, and CO₂ sensor) may be utilized to inform the operator or processor and as a result of said notification, voltage changes may be subsequently actuated to the multi-channel high voltage positive (±) regulator, targeting the highest rates of electrostatic mist deposition to the slowest growing growth trays (or support structures).

### Scope of Disclosure

It will be understood that the embodiments described herein can be implemented in various end-product applications, including food and non-food product applications. Additionally, the following non-exhaustive list provides additional fungal genera and species which may be implemented, if not otherwise inconsistent with the present disclosure.

### Implementations

In some embodiments, the present disclosure provides for an extra-particle aerial mycelium, and for methods of making an extra-particle aerial mycelium, wherein the extra-particle aerial mycelium is a growth product of a fungus. In some embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces, Wolfiporia, Ceriporiopsis, Chlorociboria, Daedalea, Daedaleopsis, Daldinia, Ganoderma, Hypoxylon, Inonotus, Lenzites, Omphalotus, Oxyporus, Phanerochaete, Phellinus, Polyporellus, Porodaedalea, Pycnoporus, Scytalidium, Stereum, Trametes or Xylaria.

In some further embodiments, the fungus is a species of the genus Bondarzewia, Ceriporiopsis, Daedalea, Daedaleopsis, Fomitopsis, Ganoderma, Inonotus, Lenzites, Omphalotus, Oxyporus, Phellinus, Polyporellus, Polyporus, Porodaedalea, Pycnoporus, Stereum, Trametes or Xylaria. In some more particular embodiments, the fungus is selected from the group consisting of Bondarzewia berkleyii, Daedalea quercina, Daedaleopsis spp., Daedaleopsis confragosa, Daedaleopsis septentrionalis, Fomitopsis spp., Fomitopsis cajanderi, Fomitopsis pinicola, Ganoderma spp., Ganoderma amboinense, Ganoderma applanatum, Ganoderma atrum, Ganoderma ibbose, Ganoderma ibbose, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum, Ganoderma weberianum, Inonotus spp., Inonotus obliqus, Inonotus hispidus, Inonotus dryadeus, Inonotus tomentosus, Lenzites betulina, Phellinus spp., Phellinus ignarius, Phellinus gilvus, Polyporus spp., Polyporus squamosus, Polyporus badius, Polyporus umbellatus, Polyporus squamosus, Polyporus tuberaster, Polyporus arcularius, Polyporus albeolaris, Polyporus radicatus, Porodaedalea pini, Pycnoporus spp., Pycnoporus spp., Pycnoporus sanguineus, Pycnoporus cinnabarinus, Stereum spp., Stereum ostea, Stereum hirsutum, Trametes spp., Trametes versicolor, Trametes elegans, Trametes suaveolens, Trametes hirsute, Trametes ibbose, Trametes ochraceae, Trametes villosa, Trametes cubensis and Trametes pubescens.

In some other embodiments, the fungus is a pigment-producing fungus of a genus selected from the group consisting of Chlorociboria, Daldinia, Hypoxylon, Phanerochaete and Scytalidium.

In yet some other embodiments, the fungus is a species of the genus Ganoderma. In some further embodiments, the fungus is Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum or Ganoderma weberianum

In yet some other embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces or Wolfiporia.

In some further embodiments, the fungus is a species of the genus Pleurotus. In some more particular embodiments, the fungus is Pleurotus albidus, Pleurotus citrinopilleatus, Pleurotus columbinus, Pleurotus cornucopiae, Pleurotus dryinus, Pleurotus djamor, Pleurotus eryngii, Pleurotus floridanus, Pleurotus nebrodensis, Pleurotus ostreatus, Pleurotus populinus, Pleurotus pulmonarius, Pleurotus sajor-caju, Pleurotus salmoneo-stramineus, Pleurotus salmonicolor or Pleurotus tuber-regium.

The various illustrative steps may be implemented as electronic hardware, computer software, or combinations of both. The interchangeability of hardware and software has been described generally, in terms of functionality, and illustrated in the various illustrative components, and steps described above. Whether such functionality is implemented in hardware or software depends upon the particular application and design constraints imposed on the overall system.

The hardware and data processing apparatus used to implement the various illustrative steps described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

A software module may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for case of describing the figures, and indicate relative positions corresponding to the orientation of the figure on a properly oriented page, and may not reflect the proper orientation of a feature as implemented.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatuses, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of, or combined with, any other aspect described. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of growing an extra-particle aerial mycelium comprising:
maintaining a growth environment configured to produce an extra-particle aerial mycelium; and
introducing a plurality of electrostatically charged mist particles into the growth environment.

2. The method of claim 1 further comprising:
providing a growth matrix comprising a nutritive substrate and a fungus; and
placing the growth matrix into the growth environment.

3. The method of claim 2, further comprising incubating the growth matrix in the growth environment for an incubation time period sufficient for the fungus to digest the nutritive substrate and produce the extra-particle aerial mycelium.

4. The method of claim 1, wherein the plurality of electrostatically charged mist particles comprises at least one of: water, fertilizers, nutrients, growth factors, hormones, disinfectants, cleaning solutions, antioxidants, pesticides, integrated pest management sprays, fine powders, microorganisms, dyes, flavonoids, and terpenes.

5. The method of claim 1, wherein introducing the plurality of electrostatically charged mist particles into the growth environment comprises introducing the plurality of electrostatically charged mist particles periodically during an incubation time period.

6. The method of claim 5, wherein introducing the plurality of electrostatically charged mist particles into the growth environment comprises introducing the plurality of electrostatically charged mist particles during a portion of the incubation time period.

7. The method of claim 1, wherein introducing the plurality of electrostatically charged mist particles into the growth environment comprises:
introducing a first portion of the plurality of electrostatically charged mist particles during a first time portion of an incubation time period, wherein the first portion of the plurality of electrostatically charged mist particles has a first charge during the first time portion; and
introducing a second portion of the plurality of electrostatically charged mist particles during a second time portion of the incubation time period, wherein the second portion of the plurality of electrostatically charged mist particles has a second charge during the second time portion.

8. The method of claim 7, wherein the second charge comprises one of: a neutral charge or an opposite charge of the first charge.

9. The method of claim 1, wherein the plurality of electrostatically charged mist particles are between 0.01 μm to 100 μm in diameter.

10. The method of claim 1, wherein introducing the plurality of electrostatically charged mist particles into the growth environment further comprises passing a stream of mist particles through a charge generator.

11. The method of claim 10, wherein the charge generator comprises an electromagnetic field.

12. The method of claim 10, further comprising generating a voltage at the charge generator, the voltage comprising between 0.01 kV to 50.0 kV.

13. The method of claim 1, wherein the growth environment is configured to maintain one or more of: a temperature, a humidity, an airflow rate, an oxygen concentration, a carbon dioxide concentration, and/or a pressure.

14. The method of claim 1, further comprising generating an airflow in the growth environment.

15. The method of claim 2, wherein the plurality of electrostatically charged mist particles is homogeneously deposited across the growth matrix or a portion thereof.

16. The method of claim 15, wherein homogeneous deposition of the plurality of electrostatically charged mist particles results in homogenous extra-particle aerial mycelium growth.

17. The method of claim 1, further comprising introducing a voltage to at least a portion of the growth matrix, thereby imparting a charge to the at least portion of the growth matrix.

18. A system for producing an extra-particle aerial mycelium, the system comprising:
a growth environment;
an electrostatically charged misting system, comprising:
an outlet configured to introduce a plurality of mist particles into the growth environment,
a charge generator disposed at a distal end of the outlet, the charge generator configured to impart an electrostatic charge to the plurality of mist particles to generate a plurality of electrostatically charged mist particles; and
a processor configured to introduce the plurality of mist particles into the growth environment.

19. The system of claim 18, further comprising a growth matrix received within the growth environment, the growth matrix comprising a nutritive substrate and a fungus.

20. The system of claim 18, further comprising the plurality of electrostatically charged mist particles, wherein the plurality of electrostatically charged mist particles comprises at least one of: water, fertilizers, nutrients, growth factors, hormones, disinfectants, cleaning solutions, antioxidants, pesticides, integrated pest management sprays, fine powders, microorganisms, dyes, flavonoids, and terpenes.

21. The system of claim 18, wherein the processor is further configured to periodically introduce the plurality of electrostatically charged mist particles.

22. The system of claim 18, wherein the processor is further configured to:
introduce a first portion of the plurality of electrostatically charged mist particles during a first time portion, wherein the first portion of the plurality of electrostatically charged mist particles have a first charge during the first time portion; and
introduce a second portion of the plurality of electrostatically charged mist particles during a second time portion, wherein the second portion of the plurality of electrostatically charged mist particles have a second charge during the second time portion.

23. The system of claim 18, wherein each of the plurality of electrostatically charged mist particles are between 0.01 μm to 100 μm in diameter.

24. The system of claim 18, wherein the outlet is further configured to pass a stream of uncharged mist particles through the charge generator.

25. The system of claim 18, wherein the charge generator comprises an electromagnetic field.

26. The system of claim 18, wherein the charge generator is configured to generate a voltage between 0.01 kV to 50.0 kV.

27. The system of claim 18, wherein the charge generator comprises one of: an induction electrostatic charging ring, a conduction charging unit, a corona charging unit, or a high velocity air sprayer.

28. The system of claim 18, wherein the growth environment is configured to maintain one or more of: a temperature, a humidity, an airflow rate, an oxygen concentration, a carbon dioxide concentration, and/or a pressure.

29. The system of claim 18, wherein the growth environment is further configured to generate airflow within the growth environment.

30. The system of claim 18, further comprising at least a portion of the growth matrix comprising a charge, wherein the charge comprises a voltage.

31. A method of growing an extra-particle aerial mycelium comprising:
maintaining a growth environment comprising a growth matrix, the growth matrix comprising a substrate and configured to produce aerial mycelium; and
introducing a plurality of electrostatically charged mist particles into the growth environment,
whereby the plurality of electrostatically charged mist particles is electrostatically charged such that each of the plurality of electrostatically charged mist particles repel one another,
wherein the plurality of electrostatically charged mist particles is sufficiently charged to allow the plurality of electrostatically charged mist particles to uniformly deposit on a surface of the growth matrix, or a portion thereof, despite airflow conditions and physical hindrances within the growth environment, and
wherein the growth matrix further comprising a target deposition surface selected from a substrate surface or an extra-particle aerial mycelium surface.

32. The method of claim 31, wherein uniform deposition of the plurality of electrostatically charged mist particles is unaffected by fluctuations in the airflow conditions in the growth environment.

33. The method of claim 32, wherein unaffected comprises monitoring the airflow conditions such that fluctuations in the airflow conditions do not affect uniform mist deposition on the target deposition surface, or portion thereof.

34. The method of claim 31, further comprising introducing a voltage to at least a portion of the growth matrix, thereby imparting a charge to the at least portion of the growth matrix.
